(19) United States
(12) Patent Application Publication
(10) Pub. No.: US 2009/0203711 A1
(43) Pub. Date: Aug. 13, 2009

(54) Inhibitors of P38 Map Kinase

(75) Inventor: David Charles Festus Moffat, Oxfordshire (GB)

Correspondence Address: BANNER & WITCOFF, LTD., 1100 13th STREET, N.W., SUITE 1200, WASHINGTON, DC, 20005-4051, US

(73) Assignee: CHROMA THERAPEUTICS LTD., Abingdon (GB)

(21) Appl. No.: 12/299,498

(22) PCT Filed: May 1, 2007
(86) PCT No.: PCT/GB2007/001583

§ 371 Date: Nov. 4, 2008

(30) Foreign Application Priority Data
May 4, 2006 (GB) 0608837.1

(51) Int. Cl. A61K 31/4418; C07D 213/72; C07D 401/12; A61K 31/496; A61P 37/00; A61P 29/00

(52) U.S. Cl. 514/253.12; 546/297; 544/360; 514/349

(57) ABSTRACT

Compounds of formula (I) are inhibitors of p38 MAP kinase, and are therefore of utility in the treatment of, inter alia, inflammatory conditions including rheumatoid arthritis and COPD:

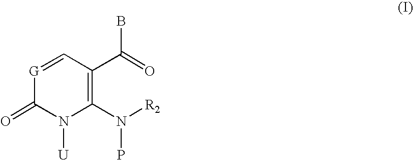

wherein: G is —N═ or —CH═; B is an optionally substituted divalent mono- or bicyclic aryl or heteroaryl radical having 5-13 ring members; R₂ is hydrogen or optionally substituted C₁-C₃ alkyl; P represents hydrogen and U represents a radical of formula (IA); or U represents hydrogen and P represents a radical of formula -A-(CH₂)_{z}-L¹-Y¹—R wherein A, z, Y¹, and L¹ are as defined in the specification; R is a radical of formula (X) or (Y)

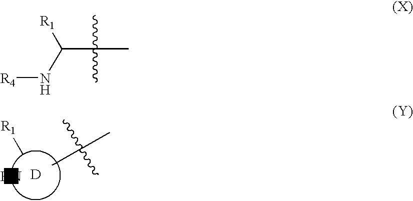

wherein R₁ is a carboxylic acid group (—COOH), or an ester group which is hydrolysable by one or more intracellular carboxylesterase enzymes to a carboxylic acid group; R₄ is hydrogen; or optionally substituted C₁-C₆ alkyl, C₃-C₇cycloalkyl, aryl or heteroaryl or —(C═O)R₃, —(C═O)OR₃, or —(C═O)NR₃ wherein R₃ is hydrogen or optionally substituted (C₁-C₆)alkyl; and D is a monocyclic heterocyclic ring of 5 or 6 ring atoms wherein R₁ is linked to a ring carbon adjacent the ring nitrogen shown, and ring D is optionally fused to a second carbocyclic or heterocyclic ring of 5 or 6 ring atoms in which case the bond shown intersected by a wavy line may be from a ring atom in said second ring.

This invention relates to a series of amino acid and amino acid ester compounds, to compositions containing them, to processes for their preparation and to their use in medicine as p38 MAP kinase inhibitors for the treatment of autoimmune and inflammatory diseases, including rheumatoid arthritis, psoriasis, inflammatory bowel disease, Crohns disease, ulcerative colitis, chronic obstructive pulmonary disease, asthma, multiple sclerosis, diabetes, atopic dermatitis, graft versus host disease, systemic lupus erythematosus and others.

### BACKGROUND OF THE INVENTION

Inappropriate activation of leukocytes including monocytes, macrophages and neutrophils leading to the production of elevated levels cytokines such as TNF-α, IL1-β and IL-8, is a feature of the pathogenesis of several inflammatory diseases including rheumatoid arthritis, ulcerative colitis, Crohn's disease, chronic obstructive pulmonary disease (COPD), asthma and psoriasis. The production of cytokines by inflammatory cells is a result of response to a variety of external stimuli, leading to the activation of a number of intracellular signalling mechanisms. Prominent amongst these is the mitogen-activated protein kinase (MAPK) superfamily consisting of highly conserved signalling kinases that regulate cell growth, differentiation and stress responses. Mammalian cells contain at least three families of MAPKs: the p42/44 extracellular signal-regulated kinase (ERK) MAPKs, c-Jun NH2-terminal kinases (JNKs) and p38 MAPK (also termed p38a/Mpk2/RK/SAPK2a/CSBP1/2). p38 MAPK was first cloned following its identification as a kinase that is tyrosine phosphorylated after stimulation of monocytes by lipopolysaccharide (LPS) [Han et al, Science 1994, 265, 808]. Additional homologues of mammalian p38 have been described and include p38, [Jiang et al, J. Biol. Chem., 1996, 271, 17920], p38γ [Li et al, Biochem. Biophys. Res. Commun., 1996, 228, 334] and p38δ [Jiang et al, J. Biol. Chem. 1997, 272, 30122]. While p38α and p38β are ubiquitously expressed, p38γ is restricted primarily to skeletal muscle and p386 is predominantly expressed in lung and kidney.

The release of cytokines by host defence cells and the response of leukocytes to cytokines and other pro-inflammatory stresses are to varying extent regulated by p38 MAPK [Cuenda et al FEBS Lett, 1995, 364, 229-233]. In other cell types, p38 MAPK controls stress responses such as the production of IL-8 by bronchial epithelial cells stimulated by TNF-α, and the up-regulation of the cell adhesion molecule ICAM-1 in LPS-stimulated endothelial cells. Upon activation, via dual phosphorylation of a TGY motif by the dual specificity kinases MKK3 and MKK6, p38 MAPK exerts its effects through phosphorylation of transcription factors and other kinases. MAP kinase-activated protein kinase-2 (MAPKAPK-2) has been identified as a target for p38 phosphorylation. It has been demonstrated that mice [Kotlyarov et al Nat. Cell Biol. 1999, 1, 94-97] lacking MAPKAP-K2 release reduced levels of TNF-α, IL-1β, IL-6, IL-10 and IFN-γ in response to LPS/galactosamine mediated endotoxic shock. The regulation of the levels of these cytokines as well as COX-2 is at the mRNA level. TNF-α levels are regulated through translational control via AU-rich elements of the 3′-UTR of TNF-α mRNA, with MAPKAP-K2 signalling increasing TNF-α mRNA translation. MAPKAP-K2 signalling leads to increased mRNA stability for COX-2, IL-6 and macrophage inflammatory protein. MAPKAP-K2 determines the cellular location of p38 MAPK as well as transducing p38 MAPK signalling, possessing a nuclear localisation signal at its carboxyl terminus and a nuclear export signal as part of its autoinhibitory domain [Engel et al, EMBO J. 1998, 17, 3363-3371]. In stressed cells, MAPKAP-K2 and p38 MAPK migrate to the cytoplasm from the nucleus, this migration only occurring when p38 MAPK is catalytically active. It is believed that this event is driven by the exposure of the MAPKAP K2 nuclear export signal, as a result of phosphorylation by p38 MAPK [Meng et al, J. Biol. Chem. 2002, 277, 37401-37405]. Additionally p38 MAPK either directly or indirectly leads to the phosphorylation of several transcription factors believed to mediate inflammation, including ATF1/2 (activating transcription factors 1/2), CHOP-10/GADD-153 (growth arrest and DNA damage inducible gene 153), SAP-1 (serum response factor accessory protein-1) and MEF2C (myocyte enhancer factor-2) [Foster et al, Drug News Perspect. 2000, 13, 488-497].

It has been demonstrated in several instances that the inhibition of p38 MAPK activity by small molecules, is useful for the treatment of several disease states mediated by inappropriate cytokine production including rheumatoid arthritis, COPD, asthma and cerebral ischemia. This modality has been the subject of several reviews [Salituro et al, Current Medicinal Chemistry, 1999, 6, 807-823 and Kumar et al, Nature Reviews Drug Discovery 2003, 2, 717-726].

Inhibitors of p38 MAPK have been shown to be efficacious in animal models of rheumatoid arthritis, such as collagen-induced arthritis in rat [Revesz et al, Biorg. Med. Chem. Lett., 2000, 10, 1261-1364] and adjuvant-induced arthritis in rat [Wadsworth et al, J. Pharmacol. Exp. Ther., 1999, 291, 1685-1691]. In murine models of pancreatitis-induced lung injury, pretreatment with a p38 MAPK inhibitor reduced TNF-α release in the airways and pulmonary edema [Denham et al, Crit. Care Med., 2000, 29, 628 and Yang et al, Surgery, 1999, 126, 216]. Inhibition of p38 MAPK before ovalbumin (OVA) challenge in OVA-sensitized mice decreased cytokine and inflammatory cell accumulation in the airways in an allergic airway model of inflammation, [Underwood et al, J. Pharmacol. Exp. Ther., 2000, 293, 281]. Increased activity of p38MAP kinase has been observed in patients suffering from inflammatory bowel disease [Waetzig et al, J. Immunol, 2002, 168, 5432-5351]. p38 MAPK inhibitors have been shown to be efficacious in rat models of cardiac hypertrophy [Behr et al, Circulation, 2001, 104, 1292-1298] and cerebral focal ischemia [Barone et al, J. Pharmacol. Exp. Ther., 2001, 296, 312-321].

We have now discovered a group of compounds which are potent and selective inhibitors of p38 MAPK (p38α,β,γ and δ) and the isoforms and splice variants thereof especially p38α, p38β and p38β2. The compounds are thus of use in medicine, for example in the treatment and prophylaxis of immune and inflammatory disorders described herein. The compounds are characterised by the presence in the molecule of an amino acid motif or an amino acid ester motif which is hydrolysable by an intracellular carboxylesterase. Compounds of the invention having the lipophilic amino acid ester motif cross the cell membrane, and are hydrolysed to the acid by the intracellular carboxylesterases. The polar hydrolysis product accumulates in the cell since it does not readily cross the cell membrane. Hence the p38 MAP kinase activity of the compound is prolonged and enhanced within the cell. The compounds of the invention are related to the p38 MAP kinase inhibitors encompassed by the disclosures in International Patent Application WO03076405 but differ therefrom in that the present compounds have the amino acid ester motif referred to above.

### DETAILED DESCRIPTION OF THE INVENTION

According to the invention there is provided a compound of formula (I):

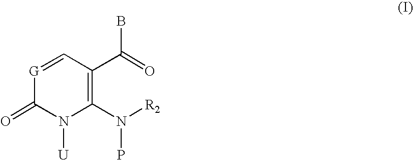

wherein:

### G is —N═ or —CH═

B is an optionally substituted divalent mono- or bicyclic aryl or heteroaryl radical having 5-13 ring members;
R₂ is hydrogen or optionally substituted C₁-C₃ alkyl;
P represents hydrogen and U represents a radical of formula (IA); or U represents hydrogen and P represents a radical of formula (IA);

-A-(CH₂)_{z}-L¹-Y¹—R (IA)

wherein
A represents an optionally substituted divalent mono- or bicyclic carbocyclic or heterocyclic radical having 5-13 ring members;
z is 0 or 1;
Y¹ is a bond, —(C═O)—, —S(O₂)—, —(C═O)NR₃—, —NR₃(C═O)—, —S(O₂)NR₃—, —NR₃S(O₂)—, or —NR₃(C═O)NR₅—, wherein R₃ and R₅ are independently hydrogen or optionally substituted (C₁-C₆)alkyl,
L¹ is a divalent radical of formula -(Alk¹)ₘ(Q)ₙ(Alk²)ₚ— wherein
m, n and p are independently 0 or 1,
Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
   R is a radical of formula (X) or (Y)

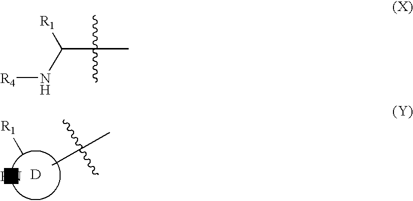

wherein
R₁ is a carboxylic acid group (—COOH), or an ester group which is hydrolysable by one or more intracellular carboxylesterase enzymes to a carboxylic acid group;
R₄ is hydrogen; or optionally substituted C₁-C₆ alkyl, C₃-C₇cycloalkyl, aryl or heteroaryl or —(C═O)R₃, —(C═O)OR₃, or —(C═O)NR₃ wherein R₃ is hydrogen or optionally substituted (C₁-C₆)alkyl; and
D is a monocyclic heterocyclic ring of 5 or 6 ring atoms wherein R₁ is linked to a ring carbon adjacent the ring nitrogen shown, and ring D is optionally fused to a second carbocyclic or heterocyclic ring of 5 or 6 ring atoms in which case the bond shown intersected by a wavy line may be from a ring atom in said second ring.

Compounds of formula (I) above may be prepared in the form of salts, especially pharmaceutically acceptable salts, N-oxides, hydrates, and solvates thereof. Any claim to a compound herein, or reference herein to “compounds of the invention”, “compounds with which the invention is concerned”, “compounds of formula (I)” and the like, includes salts, N-oxides, hydrates, and solvates of such compounds.

Although the above definition potentially includes molecules of high molecular weight, it is preferable, in line with general principles of medicinal chemistry practice, that the compounds with which this invention is concerned should have molecular weights of no more than 600.

In another broad aspect the invention provides the use of a compound of formula (I) as defined above, or an N-oxide, salt, hydrate or solvate thereof in the preparation of a composition for inhibiting the activity p38 MAP kinase enzyme.

The compounds with which the invention is concerned may be used for the inhibition of p38 MAP kinase enzyme activity in vitro or in vivo.

In one aspect of the invention, the compounds of the invention may be used in the preparation of a composition for the treatment of autoimmune or inflammatory disease, particularly those mentioned above in which p38 MAP kinase activity plays a role.

In another aspect, the invention provides a method for the treatment of the foregoing disease types, which comprises administering to a subject suffering such disease an effective amount of a compound of formula (I) as defined above.

### Terminology

The term “ester” or “esterified carboxyl group” means a group R^{X}O(C═O)— in which R^{X} is the group characterising the ester, notionally derived from the alcohol R^{X}OH.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl” wherein a and b are integers refers to straight chain or branched chain hydrocarbon groups having from a to b carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from a to b carbon atoms, and at least one triple bond.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

A “divalent phenylene, pyridinylene, pyrimidinylene, or pyrazinylene radical” is a benzene, pyridine, pyrimidine or pyrazine ring, with two unsatisfied valencies, and includes 1,3-phenylene, 1,4-phenylene, and the following:

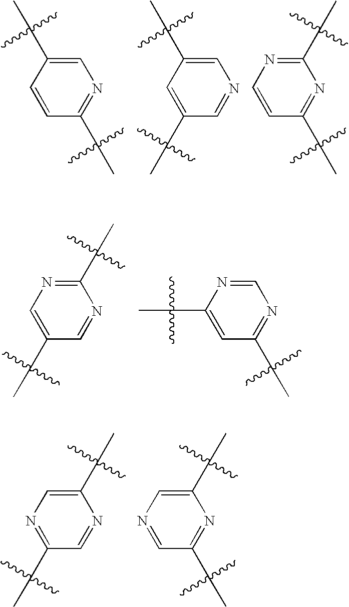

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrophalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulphonic, glutamic, lactic, and mandelic acids and the like. For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds of the invention which contain one or more actual or potential chiral centres, because of the presence of asymmetric carbon atoms, can exist as enantiomers or as a number of diastereoisomers with R or S stereochemistry at each chiral centre. The invention includes all such enantiomers and diastereoisomers and mixtures thereof.

As mentioned, the esters of the invention are converted by intracellular esterases to the carboxylic acids. Both the esters and carboxylic acids may have p38 MAP kinase inhibitory activity in their own right. The compounds of the invention therefore include not only the ester, but also the corresponding carboxylic acid hydrolysis products.

In the compounds with which the invention is concerned:

### The Group B

B is an optionally substituted divalent mono- or bicyclic aryl or heteroaryl radical having 5-13 ring members. At present it is preferred that B be optionally substituted phenyl or optionally substituted pyridinyl. Preferred optional substituents in B include chloro, fluoro, methyl, methoxy and trifluoromethyl, for example when B is 2,4-difluorophenyl.

### The Substituent R₂

R₂ is hydrogen or optionally substituted C₁-C₃ alkyl. Presently it is preferred that R₂ be hydrogen or methyl.

### P/U Regioisomers

Presently it is preferred that P be hydrogen and U be a radical of formula (IA) as defined above.

### The Radical A

In the radical of formula (IA), it is currently preferred that A be optionally substituted 1,4 phenylene. In that case preferred optional substituents include fluoro and chloro. A may also be, for example, any of the following, optionally substituted:

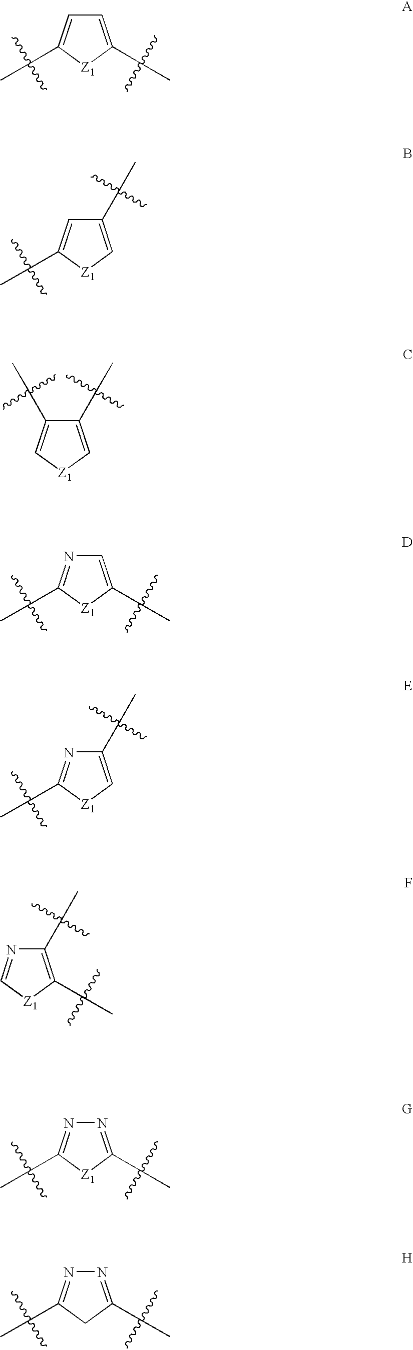

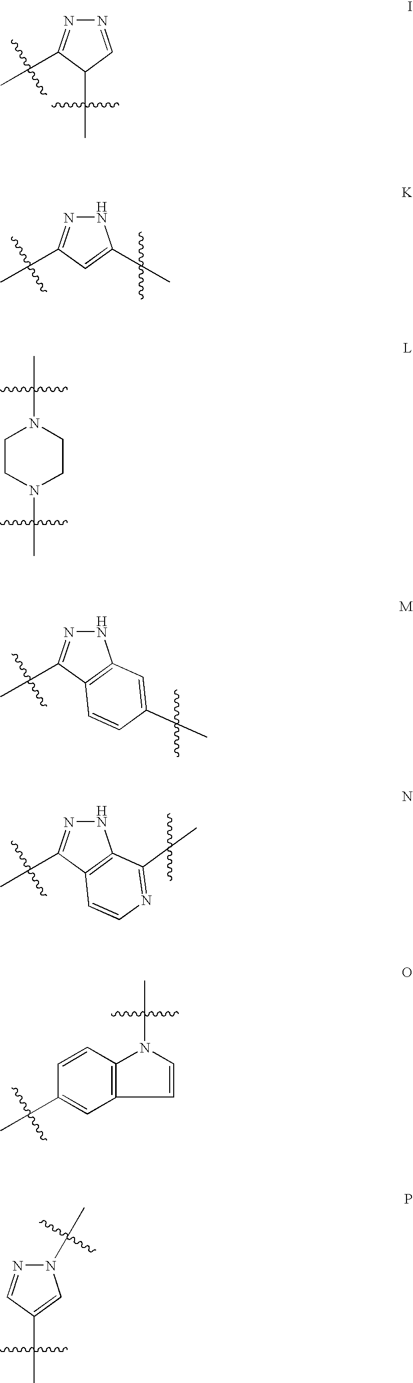

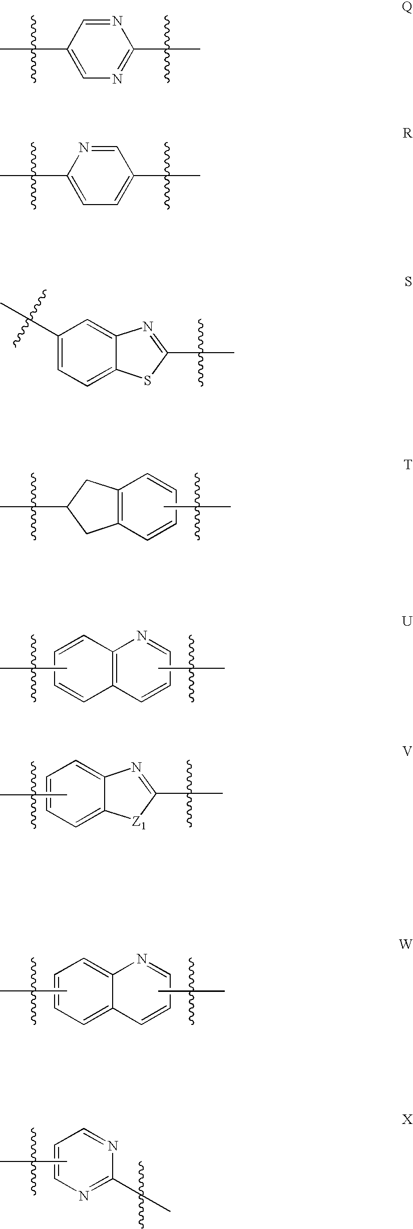

wherein Z₁ is NH, S or O.

A particularly preferred sub-group of compounds of the invention consists of those of formula (IIA), (IIB) and (IIC):

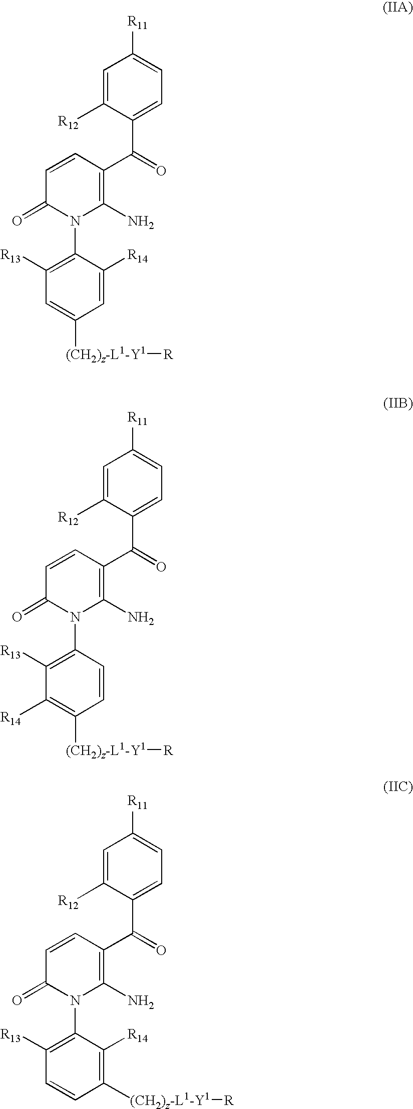

wherein
R₁₁═F, R₁₂═H, R₁₃═H and R₁₄═H; or
R₁₁═F, R₁₂═F. R₁₃═H and R₁₄═H; or
R₁₁═F, R₁₂═H, R₁₃═F and R₁₄═F; or
R₁₁═F, R₁₂═F, R₁₃═F and R₁₄═F; or
R₁₁═F, R₁₂═F, R₁₃═F and R₁₄═H.
   and wherein z, L¹, Y¹, and R are as defined above with reference to formula (I), and as further discussed below.
   The Radical —[CH₂]_{z}-L¹-Y¹—

This radical (or bond) arises from the particular chemistry strategy chosen to link the amino acid ester motif R to the ring system A. Clearly the chemistry strategy for that coupling may vary widely, and thus many combinations of the variables Y¹, L¹, and z are possible. Hence the precise combination of variable making up the linking chemistry between the amino acid ester motif and the ring system A will often be irrelevant to the primary binding mode of the compound as a whole. On the other hand, that linkage chemistry may in some cases pick up additional binding interactions with the enzyme.

With the foregoing general observations in mind, taking the variables making up the radical —[CH₂]_{z}-L¹-Y¹— in turn:
z may be 0 or 1, so that a methylene radical linked to the ring A is optional; However, in a preferred subclass of compounds of the invention z is 0.
Y¹ may be, for example, —NR₃—, —S—, —O—, —C(═O)NR₃—, —NR₃C(═O)—, or —C(═O)O—, wherein R₃ is hydrogen or optionally substituted C₁-C₆ alkyl such as —CH₂CH₂OH; In a preferred subclass of compounds of the invention, Y¹ is —O—, especially when z is 0;
In another subclass of compounds of the invention Y¹ is a bond.
In the radical L¹, examples of Alk¹ and Alk² radicals, when present, include —CH₂—, —CH₂CH₂—, —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C═CCH₂—, CH₂C═C—, and CH₂CECCH₂. Additional examples of Alk¹ and Alk² include —CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals. At present it is preferred that Alk¹ and Alk² radicals, when present, are selected from —CH₂—, —CH₂CH₂—, —CH₂CH₂CH₂—, and divalent cyclopropyl, cyclopentyl and cyclohexyl radicals.
In L¹, when n is 0, the radical is a hydrocarbon chain (optionally substituted and perhaps having an ether, thioether or amino linkage). Presently it is preferred that there be no optional substituents in L¹. When both m and p are 0, L¹ is a divalent mono- or bicyclic carbocyclic or heterocyclic radical with 5-13 ring atoms (optionally substituted). When n is 1 and at least one of m and p is 1, L¹ is a divalent radical including a hydrocarbon chain or chains and a mono- or bicyclic carbocyclic or heterocyclic radical with 5-13 ring atoms (optionally substituted). When present, Q may be, for example, a divalent phenyl, naphthyl, cyclopropyl, cyclopentyl, or cyclohexyl radical, or a mono-, or bi-cyclic heterocyclic radical having 5 to 13 ring members, such as piperidinyl, piperazinyl, indolyl, pyridyl, thienyl, or pyrrolyl radical, but 1,4-phenylene is presently preferred.
Specifically, in some embodiments of the invention, L¹, m and p may be 0 with n being 1. In other embodiments, n and p may be 0 with m being 1. In further embodiments, m, n and p may be all 0. In still further embodiments m may be 0, n may be 1 with Q being a monocyclic heterocyclic radical, and p may be 0 or 1. Alk¹ and Alk², when present, may be selected from —CH₂—, —CH₂CH₂—, and —CH₂CH₂CH₂— and Q may be 1,4-phenylene.
Examples of the radical —[CH₂]_{z}-L¹-Y¹— include —(CH₂)₃NH—, —CH₂C(═O)NH—, —CH₂CH₂C(═O)NH—, —CH₂C(O)O—, —CH₂S—, —CH₂CH₂C(O)O—, —(CH₂)₄NH—, —CH₂CH₂S—, —CH₂O, —CH₂CH₂O—, —CH₂CH₂CH₂O—

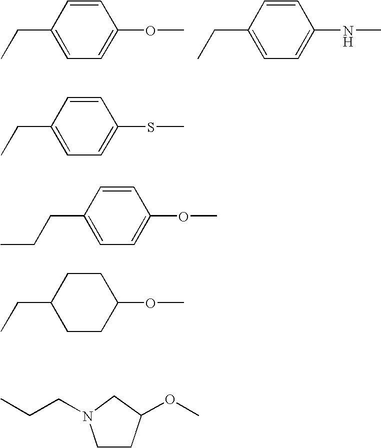

In some compounds of the invention, the radical —[CH₂]_{z}-L¹-Y¹— is —CH₂—. In other compounds of the invention, the radical —[CH₂]_{z}-L¹-Y¹— is —CH₂CH₂O— or —CH₂CH₂CH₂O—.

### The Radical R

This radical is an alpha amino acid or alpha amino acid ester moiety of formula (X) or (Y). It is linked through a linker radical -A-[CH₂]_{z}-L¹-Y¹— to the rest of the molecule.

The ester compounds of the invention are converted by intracellular esterases to the carboxylic acid. Both the esters and carboxylic acids may have p38 inhibitory activity in their own right. The compounds of the invention therefore include not only the ester, but also the corresponding carboxylic acid hydrolysis products.

### The Ester Group R₁, in the Radical R

The ester group R₁ present in radical R must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes other enzymes such as biphenylhydrolase (BPH) may also have a role in hydrolysing the conjugates. In general, if the carboxylesterase hydrolyses the free amino acid ester to the parent acid it will also hydrolyse the ester motif when covalently conjugated to the modulator. Hence, the broken cell assay described herein provides a straightforward, quick and simple first screen for esters which have the required hydrolysis profile. Ester motifs selected in that way may then be re-assayed in the same carboxylesterase assay when conjugated to the p38 inhibitor via the chosen conjugation chemistry, to confirm that it is still a carboxylesterase substrate in that background.

Subject to the requirement that they be hydrolysable by intracellular carboxylesterase enzymes, examples of particular ester groups R₁ include those of formula —(C═O)OR₇ wherein R₇ is R₈R₉R₁₀C— wherein
(i) R₈ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}— wherein a and b are independently 0 or 1 and Z¹ is —O—, —S—, or —NR₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen.

Within these classes, R₁₀ is often hydrogen. Specific examples of R₇ include methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl or methoxyethyl. Currently preferred is where R₇ is cyclopentyl.

Macrophages are known to play a key role in inflammatory disorders through the release of cytokines in particular TNFα and IL-1 (van Roon et al Arthritis and Rheumatism, 2003, 1229-1238). In rheumatoid arthritis they are major contributors to the maintenance of joint inflammation and joint destruction. Macrophages are also involved in tumour growth and development (Naldini and in Carraro Curr Drug Targets Inflamm Allergy, 2005, 3-8). Hence agents that selectively target macrophage cell proliferation could be of value in the treatment of autoimmune and other disease types. Targeting specific cell types would be expected to lead to reduced side-effects. The inventors have discovered a method of targeting p38 MAP kinase inhibitors to macrophages which is based on the observation that the way in which the esterase motif is linked to the inhibitor determines whether it is hydrolysed, and hence whether or not it accumulates in different cell types. Specifically it has been found that macrophages contain the human carboxylesterase hCE-1 whereas other cell types do not. In the general formula (I) when the nitrogen of the ester motif is substituted but not directly bonded to a carbonyl i.e. when in formula X, R₄ is not H, or a group linked to the nitrogen through a —C(═O)—, —C(═O)O— or —C(═O)NR₃— radical, or in formula Y the ring system does not directly link a —C(═O), —C(═O)O— or —C(═O)NH— radical to the nitrogen of the esterase motif, the ester will only be hydrolysed by hCE-1 and hence the inhibitors will only accumulate in macrophages.

### The Amino or Substituted Amino Group R₄, in the Radical R

The group R₄ is present in the compounds of the invention when R in formula (I) is a radical of formula (X)

As mentioned above, if the modulator is intended to act only in cell types where hCE-1 is present, such as macrophages, the amino group of the carboxylesterase motif should be directly linked to a group other than carbonyl. In such cases R₄ may be optionally substituted C₁-C₆ alkyl, C₃-C₇ cycloalkyl, aryl or heteroaryl such as monocyclic heteroaryl having 5 or 6 ring atoms, for example methyl, ethyl, n- or isopropyl, cyclopropyl, cyclopentyl, cyclohexyl, phenyl, or pyridyl. In cases where macrophage specificity is not required, R₄ may be hydrogen or —(C═O)R₃, wherein R₃ is optionally substituted C₁-C₆ alkyl such as methyl, ethyl, n- or isopropyl, or n-, iso- or sec-butyl, C₃-C₇cycloalkyl such as cyclopropyl, cyclopentyl, cyclohexyl, phenyl, pyridyl, thienyl, phenyl(C₁-C₆ alkyl)-, thienyl(C₁-C₆ alkyl)- or pyridyl(C₁-C₆ alkyl)- such as benzyl, 4-methoxyphenylmethylcarbonyl, thienylmethyl or pyridylmethyl.

### The Ring D

When R is a group of formula (Y), examples of R include:

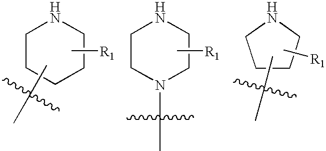

wherein R₁ is as defined and discussed above.

For compounds of the invention which are to be administered systemically, esters with a slow rate of esterase cleavage are preferred, since they are less susceptible to pre-systemic metabolism. Their ability to reach their target tissue intact is therefore increased, and the ester can be converted inside the cells of the target tissue into the acid product. However, for local administration, where the ester is either directly applied to the target tissue or directed there by, for example, inhalation, it will often be desirable that the ester has a rapid rate of esterase cleavage, to minimise systemic exposure and consequent unwanted side effects. If a carbon atom to which the group R is attached is unsubstituted, ie R is attached to a methylene (—CH₂)— radical, then the esters tend to be cleaved more rapidly than if that carbon is substituted, or is part of a ring system such as a phenyl or cyclohexyl ring.

As mentioned above, the compounds with which the invention is concerned are inhibitors of p38 MAK kinase activity, and are therefore of use in the treatment of diseases such as psoriasis, inflammatory bowel disease, Crohns disease, ulcerative colitis, chronic obstructive pulmonary disease, asthma, multiple sclerosis, diabetes, atopic dermatitis, graft versus host disease, or systemic lupus erythematosus and rheumatoid arthritis, in which p38 MAP kinase activity plays a part.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavorings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agent can be dissolved in the vehicle.

For several of the conditions treatable by compounds of the invention, one advantage lies in their property of accumulating in lung tissue, resulting in reduced systemic exposure relative to the analogous p38 MAPK inhibitor not conjugated to the amino acid ester motif. Although it is well known that agents can be given directly to the lung using inhalation methodologies, such agents still enter the systemic circulation. This can result in undesirable side effects, and can limit the dose and range of agents that can be used to treat lung disorders. Following delivery to the lung of an agent to which a hydrolysable esterase motif is attached, the neutral ester species is taken up by lung tissue where, depending on the nature of the esterase motif, it is rapidly cleaved to the acid which, as a consequence of it being a charged species, is retained in the lung tissue for a longer period of time than the neutral ester. Thus the agent is concentrated in the lung tissue and systemic exposure is reduced.

### Synthesis

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to those skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

The compounds of the invention may be prepared by a number of processes generally described below and more specifically in the Examples hereinafter. In the reactions described below, it may be necessary to protect reactive functional groups, for example hydroxyl, amino and carboxy groups, where these are desired in the final product, to avoid their unwanted participation in the reactions [see for example Greene, T. W., “Protecting Groups in Organic Synthesis”, John Wiley and Sons, 1999]. Conventional protecting groups may be used in conjunction with standard practice. In some instances deprotection may be the final step in the synthesis of a compound of general formula (I), and the processes according to the invention described herein after are understood to extend to such removal of protecting groups.

Thus compounds of general formula (I) may be prepared by, but not restricted to methods set out in Scheme 1.

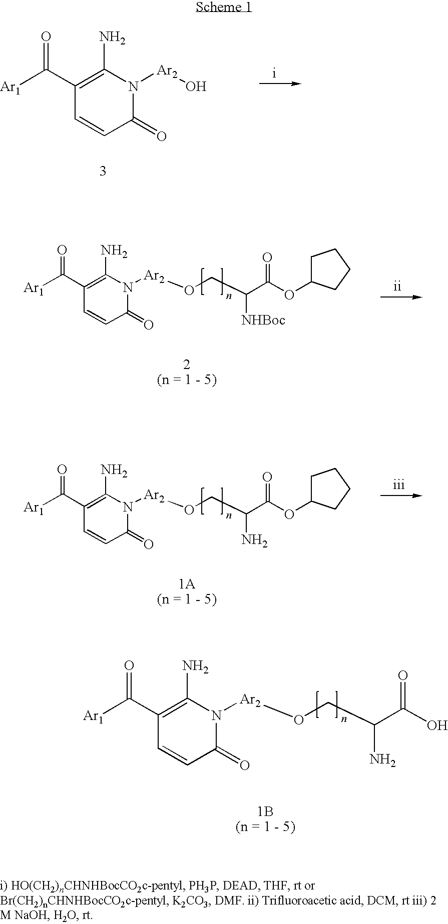

Thus compounds of general formula (1B) may be prepared by the hydrolysis of an amino acid ester of the type (1A) employing sodium hydroxide in aqueous conditions followed by acidic work-up to give the amino acid. Compounds of general formula (1A) may be prepared by treatment of tert-butyl carbamates of general formula (2) with trifluoroacetic acid in dichloromethane at ambient temperature. Compounds of general formula (2) may be prepared by the treatment of phenols of general formula (3) with, for example, N-boc protected L- or D-homoserine cyclopentyl ester in the presence of triphenyl phoshine and diethyl diazadicarboxylate in an inert ethereal solvent such as THF or diethyl ether [see for example Mitsunobu et al, Bull. Chem. Soc. Jpn., 1967, 40, 2380]. An alternative general method for preparation of compounds of formula (2) involves the alkylation of phenyl (3) with N-Boc protected L- or D-bromo homoserine cyclopentyl ester. The reaction may be performed in a dialkylamide solvent such as DMF in the presence of an inorganic base such as potassium or cesium carbonate Such reactions are set forth in March's Advanced Organic Chemistry [John Wiley and Sons, 1992]. The preparation of phenols of general formula (3) may be prepared by methods described in WO 03/076405.

In another aspect to the invention compounds of general formula (IC) and (ID) may be prepared by, but not restricted to, methods set out in Scheme 2.

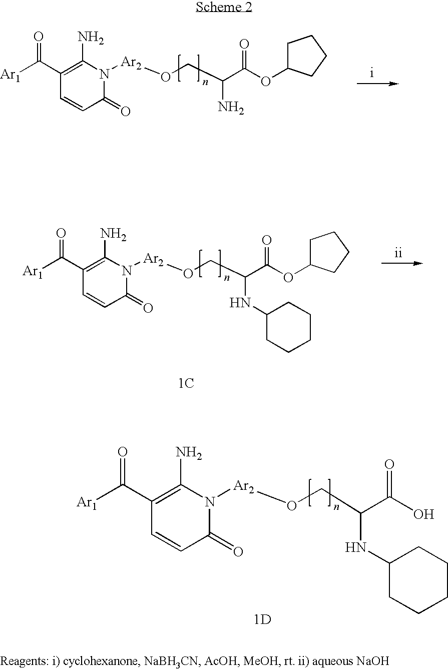

Thus, compounds of general formula (1C) may be prepared by a reductive amination process, involving the reaction of (1A) with cyclohexanone in the presence of sodium cyanoborohydride in methanol and glacial acetic acid at ambient temperature [see for example Borsch et al, J. Am. Chem. Soc., 1971, 93, 2897]. It will be recognized by those skilled in the art that this process will apply to any appropriately substituted aldehyde, ketone or cyclic ketone. Hydrolysis of esters of formula (1C) to the amino acid derivatives of formula (1D) may be performed by hydrolysis using a mineral base such as potassium or sodium hydroxide, followed by acidic work up.

In another aspect to the invention compounds of general formula (1E) and (1F) may be prepared by, but not restricted to, methods set out in Scheme 3.

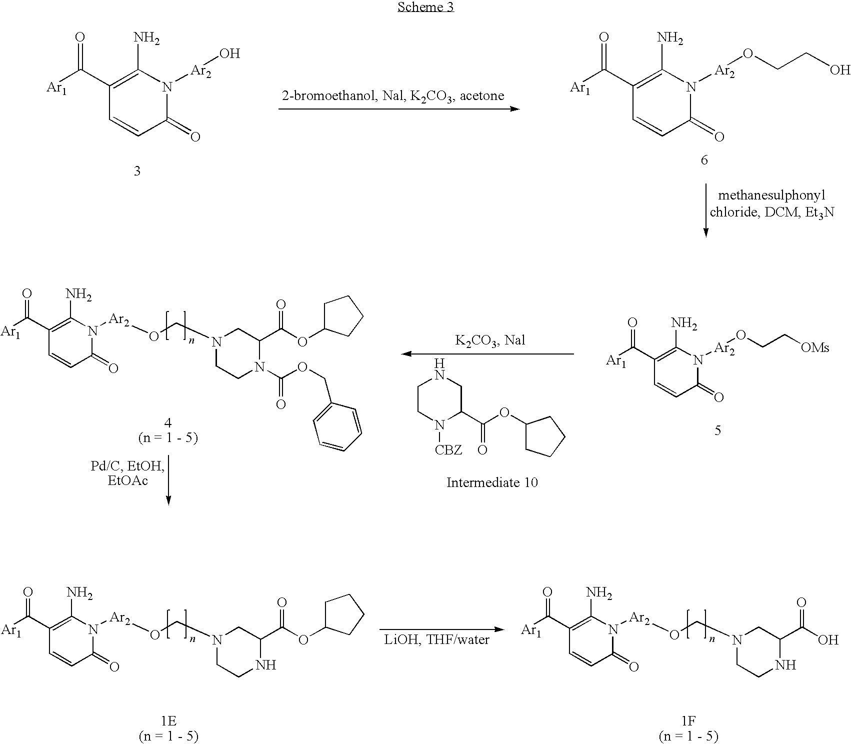

Thus compounds of general formula (1F) may be prepared by the hydrolysis of the ester of type (1E) employing lithium hydroxide in aqueous conditions followed by acidic work-up to give the carboxylic acid. Compounds of general formula (1E) may be prepared by treatment of the benzyl carbamates of general formula (4) with palladium on carbon and hydrogen gas in ethyl acetate at ambient temperature. Compounds of general formula (4) may be prepared by the alkylation of intermediates of general formula (10) with mesylates of general formula (5). The alkylation may be carried out in an inert ether solvent such as THF, in the presence of sodium iodide and inorganic bases such as potassium carbonate. Compounds of general formula (5) may be prepared by the treatment of the primary alcohol (6) with methane sulphonyl chloride in an inert solvent such as dichloromethane and in the presence of an organic base such as triethylamine.

Compounds of general formula (6) may be prepared by alkylation of phenols of general formula (3) with halo-alcohols in an inert solvent such as acetone, in the presence of sodium iodide and inorganic bases such as potassium carbonate.

The following Examples illustrate the preparation of specific compounds of the invention, and their properties: All temperatures are in ° C. The following abbreviations are used:

MeOH=methanol
EtOH=ethanol
EtOAc=ethyl acetate
Boc=tert-butoxycarbonyl
DCM=dichloromethane
DMAP=dimethylaminopyridine
DMF=dimethylformamide
DMSO=dimethyl sulfoxide
TFA=trifluoroacetic acid
THF=tetrahydrofuran
Na₂CO₃=sodium carbonate
HCl=hydrochloric acid
NaOH=sodium hydroxide
NaHCO₃=sodium hydrogen carbonate
Pd/C=palladium on carbon
TME=tert-butyl methyl ether
N₂=nitrogen
Na₂SO₄=sodium sulphate
Et₃N=triethylamine
NH₃=ammonia
TMSCl=trimethylchlorosilane
NH₄Cl=ammonium chloride
MgSO₄=magnesium sulfate
CO₂=carbon dioxide
EDCl=N-(3-Dimethylaminopropyl)-M-ethylcarbodiimide hydrochloride
Et₂O=diethyl ether
LiOH=lithium hydroxide
HOBt=1-hydroxybenzotriazole

### ELS=Evaporative Light Scattering

TLC=thin layer chromatography
ml=milliliter(s)
g=gram(s)
mg=milligram(s)
mol=moles
mmol=millimole(s)
LCMS=high performance liquid chromatography/mass spectrometry
NMR=nuclear magnetic resonance
r.t.=room temperature

Microwave irradiation was carried out using a CEM Discover focused microwave reactor. Solvents were removed using a GeneVac Series I without heating or a Genevac Series II with VacRamp at 30° C. or a Buchi rotary evaporator. Purification of compounds by flash chromatography column was performed using silica gel, particle size 40-63 μm (230-400 mesh) obtained from Silicycle. Purification of compounds by preparative HPLC was performed on Gilson systems using reverse phase ThermoHypersil-Keystone Hyperprep HS C18 columns (12 μm, 100×21.2 mm), gradient 20-100% B (A=water/0.1% TFA, B=acetonitrile/0.1% TFA) over 9.5 min, flow=30 ml/min, injection solvent 2:1 DMSO:acetonitrile (1.6 ml), UV detection at 215 nm.

¹H NMR spectra were recorded on a Bruker 400 MHz AV or a Bruker 300 MHz AV spectrometer in deuterated solvents. Chemical shifts (6) are in parts per million. Thin-layer chromatography (TLC) analysis was performed with Kieselgel 60 F₂₅₄ (Merck) plates and visualized using UV light.

Analytical HPLCMS was performed on Agilent HP1100, Waters 600 or Waters 1525 LC systems using reverse phase Hypersil BDS C18 columns (5 μm, 2.1×50 mm), gradient 0-95% B (A=water/0.1% TFA, B=acetonitrile/0.1% TFA) over 2.10 min, flow=1.0 ml/min. UV spectra were recorded at 215 nm using a Gilson G1315A Diode Array Detector, G1214A single wavelength UV detector, Waters 2487 dual wavelength UV detector, Waters 2488 dual wavelength UV detector, or Waters 2996 diode array UV detector. Mass spectra were obtained over the range m/z 150 to 850 at a sampling rate of 2 scans per second or 1 scan per 1.2 seconds using Micromass LCT with Z-spray interface or Micromass LCT with Z-spray or MUX interface. Data were integrated and reported using OpenLynx and OpenLynx Browser software.

### Intermediates

### Intermediate 1 Cyclopentyl (S)-5-{4-[6-Amino-5-(2,4-difluorobenzoyl)-2-oxo-2H-pyridin-1-yl]-3,5-difluorophenoxy}-2-tert-butoxycarbonylamino Pentanoate

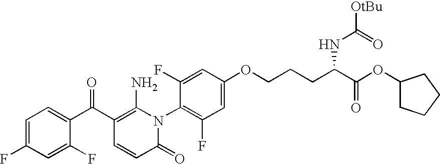

To a stirred mixture of 6-amino-5-(2,4-difluorobenzoyl)-1-(2,6-difluoro-4-hydroxy-phenyl)-1H-pyridin-2-one [prepared by methods described in WO03/076405] (100 mg, 0.265 mmol) and K2CO₃ in DMF (1.5 ml) was added cyclopentyl (2S)-5-bromo-2-[(tert-butoxycarbonyl)amino]pentanoate-[Intermediate 6] (96 mg, 0.265 mmol). The reaction mixture was stirred at 60° C. for 2 h. LCMS shows disappearance of the starting phenol, product (54%) and impurity (17%). The reaction mixture was diluted with EtOAc (15 ml) and washed sequentially with sat aq NaHCO₃ (3 ml) and water (10 ml). The EtOAc layer dried (Na₂SO₄), filtered and concentrated to dryness. Purification by flash chromatography (20% EtOAc/heptane) yielded the desired product as a white solid (50 mg, 29%). LCMS purity 100%, m/z 662 [M+H]⁺, ¹H NMR (400 MHz, MeOD), δ: 1.45 (9H, s), 1.60-2.10 (12H, m), 4.05-4.15 (3H, m), 5.15-5.25 (1H, m), 5.75 (1H, d), 6.85-6.95 (2H, m), 7.10-7.20 (2H, m), 7.40-7.60 (2H, m).

The following intermediates were prepared in an analogous manner:

### Intermediate 2 Cyclopentyl (S)-4-{4-[6-Amino-5-(2,4-difluorobenzoyl)-2-oxo-2H-pyridin-1-yl]-3,5-difluorophenoxy}-2-tert-butoxycarbonylaminobutyrate

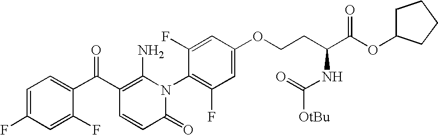

To a stirred suspension of 6-Amino-5-(2,4-difluorobenzoyl)-1-(2,6-difluoro-4-hydroxyphenyl)-1H-pyridin-2-one (100 mg, 0.26 mmol), cyclopentyl (2S)-2-[(tert-butoxycarbonyl)amino]-4-hydroxybutanoate [Intermediate 5] (83 mg, 0.29 mmol) and Ph₃P (76 mg, 0.29 mmol) in THF (0.3 ml) was added diisopropyl azodicarboxylate (0.057 ml, 0.29 mmol) dropwise. The reaction was stirred for 16 h at room temperature before concentration to dryness in vacuo. Purification by flash chromatography (100% DCM to 1% MeOH/DCM) gave the required product (132 mg) with LCMS purity 93%, m/z 648 [M+H]⁺, ¹H NMR (400 MHz, MeOD), δ: 1.30 (9H, s), 1.40-1.65 (6H, m), 1.70-1.85 (2H, m), 1.95-2.30 (2H, m), 4.00-4.10 (2H, m), 4.15-4.20 (1H, m), 5.05-5.10 (1H, m), 5.65 (1H, d), 6.70-6.80 (2H, m), 6.95-7.05 (2H, m), 7.25-7.45 (2H, m).

### Intermediate 3 t-Butyl (S)-4-{4-[6-Amino-5-(2,4-difluorobenzoyl)-2-oxo-2H-pyridin-1-yl]-3,5-difluorophenoxy}-2-{[(benzyloxy)carbonyl]amino}butyrate

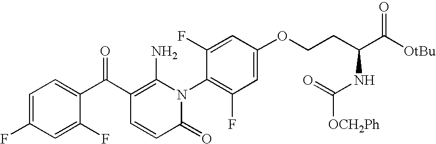

To a solution of 6-Amino-5-(2,4-difluorobenzoyl)-1-(2,6-difluoro-4-hydroxyphenyl)-1H-pyridin-2-one (100 mg, 0.26 mmol) and Intermediate 4 (108 mg, 0.29 mmol) in acetone (2 ml) was added sodium iodide (79 mg, 0.53 mmol) and potassium carbonate (146 mg, 1.06 mmol). The reaction was heated at reflux for 12 h, cooled and partitioned between water (20 ml) and ethyl acetate (20 ml). The aqueous layer was re-extracted with ethyl acetate (2×10 ml) and the combined organic extracts washed with brine (20 ml), dried (MgSO₄) and concentrated under reduced pressure to give a yellow oil. This residue was subjected to column chromatography [silica gel, 40% ethyl acetate-heptane] to give the desired product (186 mg, 79%) as a colourless solid, m/z 670 [M+H]⁺.

### Intermediate 4 Tert-Butyl (2S)-2-{[(benzyloxy)carbonyl]amino}-4-bromo Butanoate

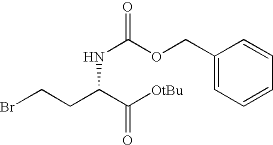

To a solution of N-bromosuccinimide (14.08 g, 79.1 mmol) in dichloromethane (100 ml) at ambient temperature was added dropwise triphenylphosphine (19.37 g, 73.9 mmol) in dichloromethane (50 ml). When addition was complete the resulting mixture was stirred for 5 min at room temperature before the dropwise addition of pyridine (2.56 ml, 31.7 mmol). To this solution was then added dropwise tert-butyl (2S)-2-{[(benzyloxy)carbonyl]amino}-4-hydroxybutanoate (8.16 g, 26.4 mmol) and the reaction stirred overnight at room temperature. The solvent was then removed under reduced pressure, with the residue being azeotroped with toluene (2×100 ml). The resulting residue was triturated with diethyl ether (250 ml) and then 10% ethyl acetate-heptane (2×100 ml). The combined organic extracts were filtered and concentrated under reduced pressure to give a yellow solid which was then subjected to column chromatography (silica, 10 to 30% ethyl acetate-heptane) to give the desired product (6.36 g) as a thick colourless oil, m/z 394/396 [M+Na]⁺.

The tert-butyl (2S)-2-{[(benzyloxy)carbonyl]amino}-4-hydroxybutanoate used as starting material was prepared as follows.

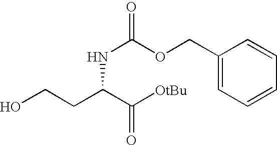

To a solution of (2S)-2-{[(benzyloxy)carbonyl]amino}succinic acid (8.54 g, 26.4 mmol) in THF (80 ml) at 0° C., was added N-methylmorpholine (4.36 ml, 39.6 mmol) and isobutylchloroformate (4.84 ml, 37.0 mmol) and resulting solution stirred for 0.5 h. Sodium borohydride (2.0 g, 52.8 mmol) was added and the reaction was continued at 0° C. for 1 h. Water (80 ml) was added and the reaction was allowed to warm to room temperature, before being extracted with ethyl acetate (3×100 ml). The combined organic extracts were washed with brine (1×100 ml), dried (MgSO₄) and concentrated in vacuo to give the desired product as a colourless oil (8.16 g) which was used without purification, m/z 332 [M+Na]⁺.

The (2S)-2-{[(benzyloxy)carbonyl]amino}succinic acid used in the above procedure was prepared as follows:

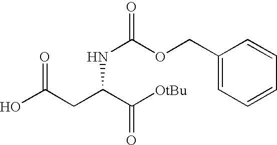

To a solution of (S)-aspartic acid t-butyl ester (5.0 g, 26 mmol) in water-dioxane (100 ml, 1:1 v/v) was added Na₂CO₃ (14.0 g, 132 mmol) slowly, followed by benzyl chloroformate (4.15 ml, 29 mmol) and the resulting mixture stirred overnight at room temperature. The reaction was then diluted with ethyl acetate (50 ml) and acidified to pH2 with concentrated HCl. The aqueous layer was separated and extracted with ethyl acetate (2×50 ml). The combined organic extracts were washed with brine, dried (MgSO₄) and concentrated under reduced pressure to give the desired product (8.54 g) as a colourless oil which was used without purification, m/z 346 [M+Na]⁺.

### Intermediate 5 Cyclopentyl (2S)-4-hydroxy-2-[(tert-butoxycarbonyl)amino]butanoate

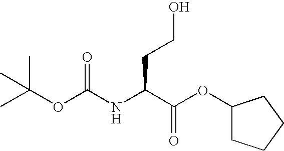

Cyclopentyl N-(tert-butoxycarbonyl)-O—[tert-butyl(dimethyl)silyl]-L-homoserinate (1.57 g, 3.9 mmol) was dissolved in acetic acid:THF:water (3:1:1, 100 ml). The reaction mixture was stirred at 30° C. for 16 hours for complete reaction. Ethyl acetate (200 ml) was added and washed with 1M Na₂CO₃, 1M HCl and brine. The ethyl acetate extracts were dried over magnesium sulphate and evaporated under reduced pressure to give the product as a clear oil which crystallised on standing (1.0 g, 95%).

LCMS purity 100%, m/z 310.3 [M+Na]⁺, ¹H NMR (250 MHz, CDCl₃), δ: 5.4 (1H, d, J=6.5 Hz), 5.2 (1H, m), 4.4 (1H, m), 3.65 (2H, m), 2.15 (1H, m), 1.9-1.55 (9H, bm), 1.45 (9H, s).

The cyclopentyl N-(tert-butoxycarbonyl)-O-[tert-butyl(dimethyl)silyl]-L-homoserinate used in the above process was prepared as follows:

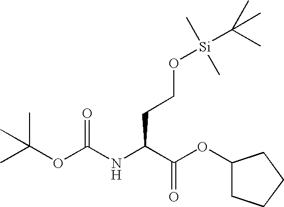

To a solution of (S)-2-tert-Butoxycarbonylamino-4-(tert-butyl-dimethyl-silanyloxy)-butyric acid (2.53 g, 7.6 mmol) in DCM (50 ml) at 0° C. was added cyclopentanol (1.39 ml, 15.3 ml, 2 eq), EDC (1.61 g, 8.4 mmol, 1.1 eq) and DMAP (0.093 g, 0.76 mmol, 0.1 eq). The reaction mixture was stirred for 16 hours at room temperature before evaporation under reduced pressure. The crude residue was dissolved in ethyl acetate (100 ml) and washed with 1M HCl, 1M Na₂CO₃ and brine. The organic layer was then dried over magnesium sulphate and evaporated under reduced pressure. The product was purified by column chromatography using ethyl acetate/heptane (1:4) to give 2.24 g, 73% yield of title compound.

LCMS purity 100%, m/z 402.5 [M+H]⁺, ¹H NMR (300 MHz, CDCl₃), δ: 5.2 (1H, d, J=6.3 Hz), 5.15 (1H, m), 4.2 (1H, m), 3.6 (2H, m), 2.0 (1H, m), 1.95-1.55 (9H, bm), 1.4 (9H, s), 0.85 (9H, s), 0.1 (6H, s).

The (S)-2-tert-Butoxycarbonylamino-4-(tert-butyl-dimethyl-silanyloxy)-butyric acid used in the above process was prepared as follows:

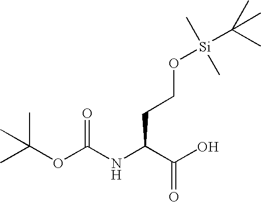

The suspension of (S)-2-amino-4-(tert-butyldimethylsilanyloxy)butyric acid (1.8 g, 7.7 mmol) in DCM (100 ml) at 0° C. was treated with triethylamine (2.15 ml, 15.4 mmol, 2 eq) and di-tert-butyl dicarbonate (1.77 g, 8.1 mmol, 1.05 eq). The reaction mixture was stirred at room temperature for 16 hours for complete reaction. The DCM was removed under reduced pressure and the mixture was treated with ethyl acetate/brine. The ethyl acetate layer was dried over magnesium sulphate and evaporated under reduced pressure. The crude product was taken forward without further purification (2.53 g, 99%). ¹H NMR (400 MHz, CDCl₃), δ: 7.5 (1H, bs), 5.85 (1H, d, J=6.5 Hz), 4.3 (1H, m), 3.75 (2H, m), 1.95 (2H, m), 1.40 (9H, s), 0.85 (9H, s), 0.1 (6H, s).

The (S)-2-amino-4-(tert-butyldimethylsilanyloxy)butyric acid used in the above process was prepared as follows:

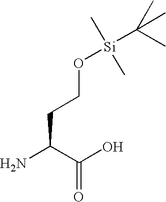

To a suspension of L-Homoserine (1 g, 8.4 mmol) in acetonitrile (10 ml) at 0° C. was added 1,8-Diazabicyclo[5.4.0]undec-7-ene (1.32 ml, 8.8 mmol, 1.05 eq). Tert-butyl-dimethylsilyl chloride (1.33 g, 8.8 mmol, 1.05 eq) was then added portionwise over 5 minutes and the reaction mixture allowed to warm to room temperature and stirred for 16 hours. A white precipitate had formed which was filtered off and washed with acetonitrile before drying under vacuum. The title compound was isolated as a white solid (1.8 g, 92%). ¹H NMR (400 MHz, DMSO), 6: 7.5 (1H, bs), 3.7 (1H, m), 3.35 (4H, bm), 1.95 (1H, m), 1.70 (1H, m), 0.9 (9H, s), 0.1 (6H, s).

### Intermediate 6 Cyclopentyl (2S)-5-bromo-2-[(tert-butoxycarbonyl)amino]pentanoate

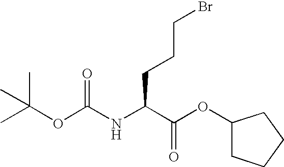

To a slurry of N-bromo succinimide (3.54 g, 19.9 mmol, 3 eq) in DCM (30 ml) was added a solution of triphenyl phosphine (4.87 g, 18.8 mmol, 2.8 eq) in DCM (15 ml). The solution was stirred for a further 5 minutes before addition of pyridine (644 μl, 7.96 mmol, 1.2 eq) and a solution of cyclopentyl (2S)-5-hydroxy-2-[(tert-butoxycarbonyl)amino]pentanoate (2.0 g, 6.64 mmol) in DCM (20 ml). The solution was stirred for 18 hrs, concentrated in vacuo and the residual solvent azeotroped with toluene (3×30 ml). The residue was triturated with diethyl ether (30 ml) and ethyl acetate:heptane (1:9, 2×30 ml). The combined ether and ethyl acetate/heptane solutions was concentrated onto silica and purified by column chromatography using ethyl acetate/heptane (1:9-2:8) to provide 1.34 g (55% yield) of title compound as a clear oil.

¹H NMR (300 MHz, CDCl₃), δ: 5.25 (1H, m), 5.05 (1H, bd), 3.45 (2H, m), 2.00-1.55 (12H, bm), 1.45 (9H, s).

The cyclopentyl (2S)-5-hydroxy-2-[(tert-butoxycarbonyl)amino]pentanoate used as starting material in the above process was prepared as follows:

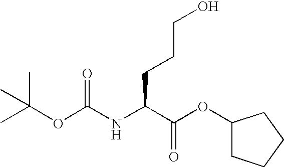

Ethyl chloroformate (2.45 ml, 25.6 mmol, 1.2 eq) was added at −20° C. to a stirred solution of (S)-2-tert-Butoxycarbonylamino-pentanedioic acid 1-cyclopentyl ester (6.73 g, 21.4 mmol) and N-methyl morpholine (3.05 ml, 27.8 mmol, 1.3 eq) in THF (50 ml). The reaction mixture became very thick with precipitation of a white solid. The reaction was therefore diluted further with THF (100 ml) to aid mixing and left stirring at −20° C. for 2 hours. The precipitated mass was filtered off and the filtrate was added over a period of 20 minutes to a solution of sodium borohydride (2.43 g, 64.1 mmol, 3 eq) in THF (20 ml) and water (5 ml) at 0° C. The reaction mixture was allowed to stir to room temperature and left for 4 hours for complete reaction. The mixture was acidified to pH 5 with 1M HCl and the THF removed under reduced pressure. The aqueous solution was extracted with EtOAc (3×100 ml) and dried over magnesium sulphate. The product was purified by column chromatography (DCM-5% MeOH/DCM) and isolated as a clear oil (5.0 g, 78%). ¹H NMR (300 MHz, CDCl₃), δ: 5.20 (2H, m), 4.25 (1H, m), 3.65 (2H, m), 2.00-1.57 (12H, bm), 1.47 (9H, s).

The (S)-2-tert-Butoxycarbonylamino-pentanedioic acid 1-cyclopentyl ester used as starting material in the above process was prepared as follows:

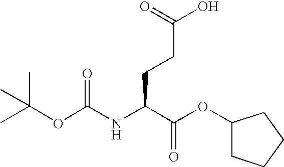

(S)-2-tert-Butoxycarbonylamino-pentanedioic acid 5-benzyl ester 1-cyclopentyl ester (12.4 g, 30.5 mmol) was dissolved in EtOAc (200 ml) and purged with nitrogen before addition of 20% Pd(OH)₂ on carbon catalyst (1.3 g). The reaction flask was then purged with hydrogen gas for a period of 5 minutes before leaving under a balloon of hydrogen for 5 hours for complete reaction. The catalyst was removed by filtration, washing with 50 ml EtOAc and the combined mother liquors were evaporated under reduced pressure. The title compound was isolated as a clear oil (7.73 g, 85%) and required no further purification. ¹H NMR (300 MHz, CDCl₃), δ: 10.0 (1H, bs), 5.70 (2H, m), 4.28 (1H, m), 2.47 (2H, m), 2.15 (1H, m), 1.95-1.55 (9H, bm), 1.47 (9H, s).

The (S)-2-tert-Butoxycarbonylamino-pentanedioic acid 5-benzyl ester 1-cyclopentyl ester used as starting material in the above process was prepared as follows:

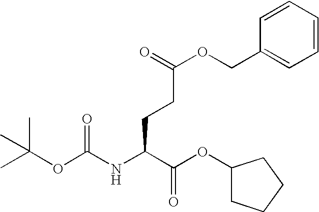

To a solution of Boc-L-Glu(OBzl)-OH (15 g, 44.5 mmol) in dichloromethane (220 ml) in an ice-bath, was added cyclopentanol (4.8 ml, 53.3 mmol, 1.2 eq), EDC (9.4 g, 48.9 mmol, 1.1 eq) and DMAP (543 mg, 4.4 mmol, 0.1 eq). The reaction mixture was allowed to warm to room temperature and stirred for 12 hours for complete reaction. The reaction mixture was diluted with DCM (200 ml) and washed with 1M HCl, 1M Na₂CO₃ and brine. The organic layer was then dried over magnesium sulphate and evaporated under reduced pressure. The product was purified by column chromatography using ethyl acetate/heptane (1:4) to give 12.4 g, 69% yield of title compound as a white solid. ¹H NMR (300 MHz, CDCl₃), δ: 7.38 (5H, m), 5.70 (1H, m), 5.10 (2H, s), 5.05 (1H, m), 4.25 (1H, m), 2.47 (2H, m), 2.15 (1H, m), 1.95-1.55 (9H, bm), 1.47 (9H, s).

### Intermediate 7 Tert-Butyl (2S)-2-{[(benzyloxy)carbonyl]amino}-5-bromo Pentanoate

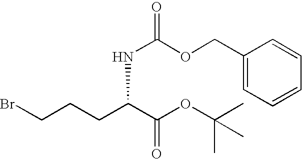

Intermediate 7 was prepared using similar methodology to Intermediate 4, starting from (S)-glutamic acid t-butyl ester. m/z 409 [M+Na]⁺.

### Intermediate 8 Tert-Butyl (S)-4-{4-[6-Amino-5-(2,4-difluorobenzoyl)-2-oxo-2H-pyridin-1-yl]-3,5-difluorophenoxy}-2-{[(benzyloxy)carbonyl]amino}pentanoate

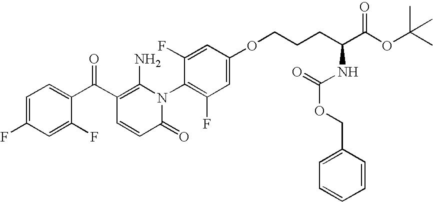

To a solution of 6-Amino-5-(2,4-difluorobenzoyl)-1-(2,6-difluoro-4-hydroxyphenyl)-1H-pyridin-2-one (100 mg, 0.26 mmol) and Intermediate 7 (108 mg, 0.29 mmol) in acetone (2 ml) was added sodium iodide (79 mg, 0.53 mmol) and potassium carbonate (146 mg, 1.06 mmol). The reaction was heated at reflux for 12 h, cooled and partitioned between water (20 ml) and ethyl acetate (20 ml). The aqueous layer was re-extracted with ethyl acetate (2×10 ml) and the combined organic extracts washed with brine (20 ml), dried (MgSO₄) and concentrated under reduced pressure to give a yellow oil. This residue was subjected to column chromatography [silica gel, 40% ethyl acetate-heptane] to give the desired product (161 mg, 89%) as a colourless solid, m/z 684 [M+H]+

### Intermediate 9 1-Benzyl 2-cyclopentyl 4-(2-{4-[6-amino-5-(2,4-difluorobenzoyl)-2-oxopyridin-1(2H)-yl]-3,5-difluorophenoxy}ethyl)piperazine-1,2-dicarboxylate

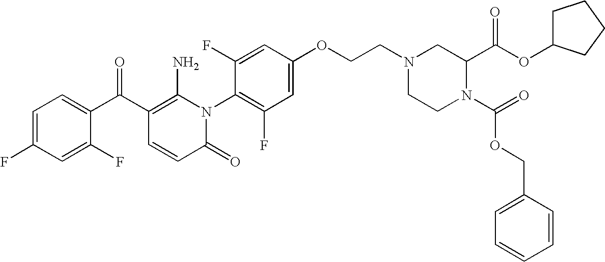

To a solution of 2-{4-[6-amino-5-(2,4-difluorobenzoyl)-2-oxopyridin-1 (2H)-yl]-3,5 difluorophenoxy}ethyl methanesulfonate (118 mg, 0.24 mmol) and Intermediate 10 (117 mg, 0.35 mmol) in THF (4 ml) was added sodium iodide (72 mg, 0.48 mmol) and potassium carbonate (66 mg, 0.48 mmol). The reaction was heated to 70° C. for 12 h, cooled and partitioned between water (20 ml) and ethyl acetate (20 ml). The aqueous layer was re-extracted with ethyl acetate (2×10 ml) and the combined organic extracts washed with brine (20 ml), dried (MgSO₄) and concentrated under reduced pressure to give a yellow oil. This residue was subjected to column chromatography [silica gel, 0-80% ethyl acetate-heptane] to give the desired product (40 mg, 23%) as a colourless solid, m/z 737 [M+H]⁺

### Intermediate 9a 2-{4-[6-amino-5-(2,4-difluorobenzoyl)-2-oxopyridin-1(2H)-yl]-3,5-difluorophenoxy}ethyl Methanesulfonate

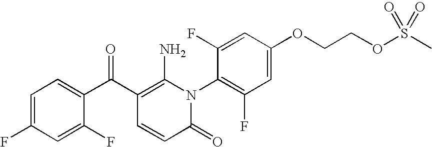

To a solution of 6-Amino-5-(2,4-difluoro-benzoyl)-1-[2,6-difluoro-4-(2-hydroxy-ethoxy)-phenyl]-1H-pyridin-2-one (120 mg, 0.28 mmol) in DCM (6 ml) at 0° C. was added methanesulphonyl chloride (24.2 μl, 0.31 mmol) and triethylamine (78 μl, 0.56 mmol). Reaction was stirred for 1 hour before the addition of a further 12 μl of methane sulphonyl chloride. Reaction allowed to stir for 12 hours for complete reaction. The reaction was diluted with DCM (10 ml) and washed with 10% citric acid, aqueous NaHCO₃, and brine. The organic layer was dried (MgSO₄) and concentrated under reduced pressure. The product was taken forward without any further purification (118 mg, 84%).

The 6-Amino-5-(2,4-difluoro-benzoyl)-1-[2,6-difluoro-4-(2-hydroxy-ethoxy)-phenyl]-1H-pyridin-2-one used in the above process was prepared as shown below.

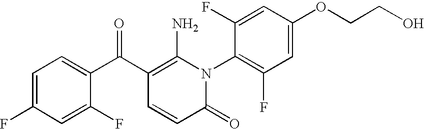

6-Amino-5-(2,4-difluoro-benzoyl)-1-(2,6-difluoro-4-hydroxy-phenyl)-1H-pyridin-2-one (400 mg, 1.06 mmol), 2-bromoethanol (82 μl, 1.16 mmol), NaI (314 mg, 2.12 mmol), K2CO₃ (586 mg, 4.24 mmol) in acetone (20 ml) was heated to 70° C. for 12 h, cooled and partitioned between water (20 ml) and ethyl acetate (20 ml). The aqueous layer was re-extracted with ethyl acetate (2×10 ml) and the combined organic extracts washed with brine (20 ml), dried (MgSO₄) and concentrated under reduced pressure to give a yellow oil. This residue was subjected to column chromatography [silica gel, 0-50% ethyl acetate-heptane] to give the desired product (120 mg, 27%) as a white solid, m/z 423 [M+H]⁺

### Intermediate 10 1-benzyl 2-cyclopentyl piperazine-1,2-dicarboxylate

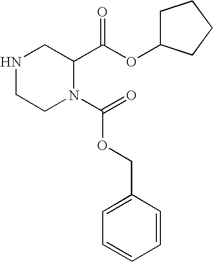

1-benzyl 4-tert-butyl 2-cyclopentyl piperazine-1,2,4-tricarboxylate (300 mg, 0.69 mmol) was dissolved in 1:1 TFA/DCM (5 ml) and stirred at room temperature for 1 hour for complete reaction. The solvent was removed under reduced pressure and the crude residue partitioned between ethyl acetate and 1M NaHCO₃. The organic layer was further washed with brine and dried over magnesium sulfate, filtered and evaporated to dryness under reduced pressure. The product was isolated as a colourless oil (220 mg, 96%).

The 1-benzyl 4-tert-butyl 2-cyclopentyl piperazine-1,2,4-tricarboxylate used as starting material in the above process was prepared as follows:

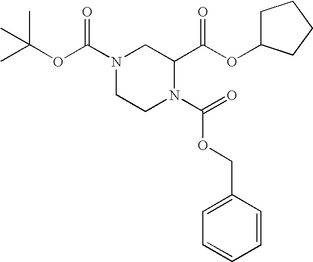

To a solution of N-Boc-N-Cbz-piperazine carboxylic acid (1 g, 2.74 mmol) in DCM (80 ml) was added cyclopentanol (498 μl, 5.49 mmol), DMAP (33 mg, 0.27 mmol) and EDCl (525 mg, 2.74 mmol). Reaction was stirred at room temperature for 12 hours. The reaction was diluted with DCM (100 ml) and washed with 1M HCl, 1M Na₂CO₃ and brine, dried over magnesium sulfate, filtered and evaporated under reduced pressure. This residue was subjected to column chromatography [silica gel, 0-20% ethyl acetate-heptane] to give the desired product (990 mg, 83%) as a colourless oil, m/z 433 [M+H]⁺

### Intermediate 11 1-Benzyl 2-cyclopentyl 4-[2-(4-{6-amino-5-[(4-fluorophenyl)carbonyl]-2-oxopyridin-1(2H)-yl}phenyl)ethyl]piperazine-1,2-dicarboxylate

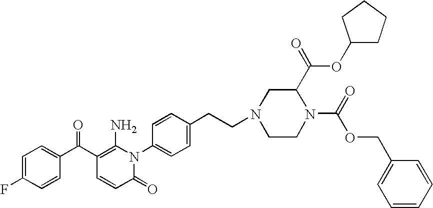

To a solution of 1-benzyl 2-cyclopentyl piperazine-1,2-dicarboxylate (114 mg, 0.31 mmol, 1.2 eq), K2CO₃ (78 mg, 0.51 mmol, 2 eq) and NaI (77 mg, 0.51 mmol, 2 eq) in THF (1 ml) under an atmosphere of nitrogen was added methanesulfonic acid 2-{4-[6-amino-5-(4-fluoro-benzoyl)-2-oxo-2H-pyridin-1-yl]-phenyl}-ethyl ester (111 mg, 0.26 mmol, 1 eq) as a solution in DMF (1 ml). The mixture was heated at 70° C. for 24 hours, before being allowed to cool to room temperature and dissolved in EtOAc (20 ml). The organic layer was washed with water (2×20 ml), dried over MgSO₄, filtered and concentrated under reduced pressure. Purification by column chromatography (3-4% MeOH in DCM) afforded an impure yellow solid containing the title compound that was used without further purification (112 mg). LC/MS: m/z 667 [M+H]⁺.

### Intermediate 11a Methanesulfonic Acid 2-{4-[6-amino-5-(4-fluoro-benzoyl)-2-oxo-2H-pyridin-1-yl]-phenyl}-ethyl Ester

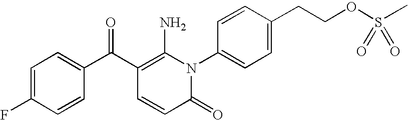

To a suspension of 6-Amino-5-(4-fluoro-3-methyl-benzoyl)-1-[4-(2-hydroxy-ethyl)-phenyl]-1H-pyridin-2-one (150 mg, 0.43 mmol) in anhydrous DCM (3 ml) at 0° C. was added methanesulfonyl chloride (34 μl, 0.47 mmol) followed by Et₃N (120 μl, 0.85 mmol). The reaction mixture was allowed to warm up to RT and stirred for 24 hours to completion. The reaction mixture was diluted with DCM (10 ml), washed with 10% citric acid (5 ml), followed by sat aq NaHCO₃ (5 ml) and water (5 ml). The DCM layer was dried (MgSO₄), filtered and concentrated in vacuo. Yield=183 mg (crude). LCMS purity=85% m/z=431 [M+H]⁺. This material was used in the next step without further purification. The alcohol used as starting material was prepared as follows:

Acetic acid 2-{4-[6-amino-5-(4-fluoro-benzoyl)-2-oxo-2H-pyridin-1-yl]-phenyl}-ethyl ester (300 mg) was dissolved in water (5 ml) and conc HCl (5 ml) and heated to 100° C. for 1 hour. The reaction was then cooled, diluted with 10 ml water and filtered. The resulting solid was then dried under reduced pressure to give 264 mg of product, m/z=353 [M+H]⁺.

The acetic acid 2-{4-[6-amino-5-(4-fluoro-benzoyl)-2-oxo-2H-pyridin-1-yl]-phenyl}-ethyl ester used as starting material was prepared as follows:

A solution of propiolic acid (270 μl, 4.39 mmol) and CDI (712 mg, 4.34 mmol) in THF (13 ml) was warmed from 0° C. to RT and stirred for 1.5 hours. To this solution was added acetic acid 2-(4-{[3-(4-fluoro-phenyl)-3-oxo-propionimidoyl]-amino}-phenyl)-ethyl ester (1 g, 2.92 mmol) in THF (6 ml) and the reaction heated to 80° C. for a period of 2 hours maximum. After cooling and evaporation under reduced pressure, the crude residue was sonicated with methanol (7 ml) before filtration, washing with a minimum amount of methanol. An off-white solid was collected (350 mg crude).

The acetic acid 2-(4-{[3-(4-fluoro-phenyl)-3-oxo-propionimidoyl]-amino}-phenyl)-ethyl ester used as starting material was prepared as follows:

3-(4-Fluoro-phenyl)-3-oxo-thiopropionimidic acid 4-chloro-phenyl ester (1 g, 2.9 mmol) and 4-aminophenethyl alcohol (418 mg, 3.08 mmol) were dissolved in acetic acid (5 ml) and heated to 80° C. for a period of 24 hours. The reaction was cooled to RT and evaporated under reduced pressure. The crude residue was partitioned between DCM and Na₂CO₃. The DCM layer was further washed with brine and dried over MgSO₄ before evaporation under reduced pressure. The product was isolated (1 g crude) as a 3:1 mixture of the acetylated product: alcohol. This was taken through unpurified into the above cyclisation reaction. Product m/z=343 [M+H]⁺, alcohol m/z=301 [M+H]⁺.

### EXAMPLES

### Example 1 Tert-Butyl (S)-2-amino-4-{4-[6-amino-5-(2,4-difluorobenzoyl)-2-oxo-2H-Pyridin-1-yl]-3,5-difluorophenoxy}butanoate

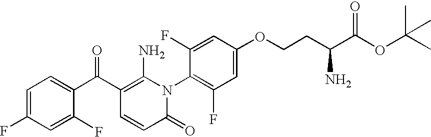

Intermediate 3 (140 mg, 0.2 mmol) was dissolved in ethyl acetate (15 ml) containing 10% palladium hydroxide on carbon (20 mg) and stirred under a hydrogen atmosphere (1 atm) for 1 h. The reaction mixture was purged with N₂, and filtered through Celite® washing with additional ethyl acetate. The filtrate was concentrated under reduced pressure to give a solid which was subjected to column chromatography [silica gel: 5% MeOH in dichloromethane]. This gave the desired product (60 mg, 54%) as a grey solid: LCMS purity 98%, m/z 536 [M+H]⁺, ¹H NMR (300 MHz, CDCl₃), δ: 7.65-7.44 (1H, m), 7.39-7.29 (2H, m), 6.96-6.82 (2H, m), 6.66 (2H, br d, J=8.1 Hz), 5.82 (1H, d, J=9.9 Hz), 4.20-4.07 (3H, m), 3.48 (1H, dd, J=4.8, 8.7 Hz), 2.22-2.15 (1H, m), 1.91-1.84 (1H, m), 1.62 (2H, br s), 1.43 (9H, s).

### Example 2 Cyclopentyl (S)-2-Amino-5-{4-[6-amino-5-(2,4-difluorobenzoyl)-2-oxo-2H-pyridin-1-yl]-3,5-difluorophenoxy}pentanoate Trifluoroacetate

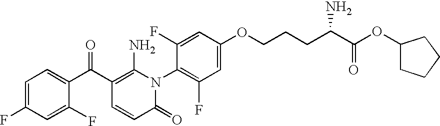

A mixture of Intermediate 1 (10 mg) and 20% TFA/DCM (0.5 ml) was allowed to stand at RT for 3 h. The reaction mixture was concentrated to dryness by blowing under N₂. The residue was triturated with Et₂O (0.3 ml×2) to give a white precipitate (9.3 mg, 91%). LCMS purity 98%, m/z 562 [M+H]⁺, ¹H NMR (400 MHz, MeOD), δ: 1.65-2.25 (12H, m), 4.15-4.25 (3H, m), 5.35-5.45 (1H, m), 5.85 (1H, d,), 6.90-7.00 (2H, m), 7.15-7.25 (2H, m), 7.50-7.65 (2H, m).

The following compounds were prepared in an analogous manner to the compound of Example 2.

### Example 3 Cyclopentyl (S)-2-Amino-4-{4-[6-amino-5-(2,4-difluorobenzoyl)-2-oxo-2H-pyridin-1-yl]-3,5-difluorophenoxy}butanoate Trifluoroacetate

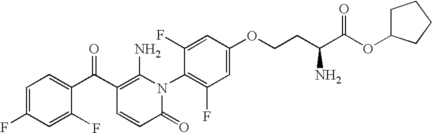

From Intermediate 2. LCMS purity 100%, m/z 548 [M+H]⁺, ¹H NMR (400 MHz, MeOD), δ: 1.55-1.80 (6H, m), 1.85-2.00 (2H, m), 2.30-2.50 (2H, m), 4.15-4.30 (3H, m), 5.25-5.35 (1H, m), 5.75 (1H, d), 6.85-6.95 (2H, m), 7.05-7.15 (2H, m), 7.40-7.55 (2H, m).

### Example 4 Cyclopentyl (R)-2-Amino-4-{4-[6-amino-5-(2,4-difluorobenzoyl)-2-oxo-2H-pyridin-1-yl]-3,5-difluorophenoxy}butanoate Trifluoroacetate

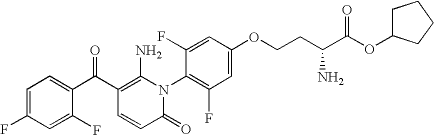

Example 4 is the (R)-enantiomer of Example 3, synthesised via the (R)-enantiomer of Intermediate 2, originating from (R)-homoserine. LCMS purity 99%, m/z 548 [M+H]⁺, ¹H NMR (400 MHz, MeOD), δ: 1.55-1.95 (8H, m), 2.25-2.45 (2H, m), 4.15-4.25 (3H, m), 5.20-5.30 (1H, m), 5.75 (1H, d), 6.75-6.90 (2H, m), 7.00-7.10 (2H, m), 7.35-7.50 (2H, m).

### Example 5 Cyclopentyl (S)-4-{4-[6-Amino-5-(2,4-difluorobenzoyl)-2-oxo-2H-pyridin-1-yl]-3,5-difluorophenoxy}-2-N-cyclohexylaminobutanoate

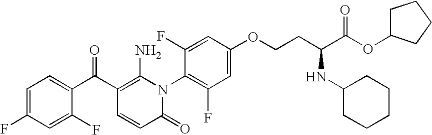

To a mixture of the compound of Example 3 (80 mg, 0.121 mmol) and K2CO₃ (25 mg, 0.18 mmol) in THF (0.5 ml) was added cyclohexanone (0.062 ml, 0.60 mmol) followed by MeOH (0.5 ml). The reaction mixture was adjusted to pH 5-6 using glacial AcOH (dropwise), stirred for 1 h before addition of NaCNBH₃ (30 mg, 0.48 mmol). Stirring at RT was continued for 18 h. The reaction mixture was concentrated to dryness by blowing under N₂, partitioned between EtOAc (5 ml) and sat aq NaHCO₃ (5 ml). EtOAc layer was dried (Na₂SO₄), filtered and concentrated to dryness under reduced pressure. Purification by preparative TLC (4% MeOH/DCM, R_{f}=0.4) gave the desired product (33 mg, 44%). LCMS purity 94%, m/z 630 [M+H]⁺, ¹H NMR (400 MHz, MeOD), δ: 0.90-1.25 (6H, m), 1.45-1.85 (12H, m), 1.90-2.10 (2H, m), 2.25-2.35 (1H, m), 3.45-3.55 (1H, m), 4.00-4.20 (2H, m), 5.10-5.20 (1H, m), 5.70 (1H, d), 6.75-6.85 (2H, m), 7.00-7.10 (2H, m), 7.35-7.45 (2H, m).

### Example 6 Tert-Butyl (S)-2-Amino-5-{4-[6-amino-5-(2,4-difluorobenzoyl)-2-oxo-2H-Pyridin-1-yl]-3,5-difluorophenoxy}pentanoate

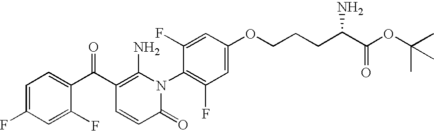

Example 6 was synthesised using the same methodology as Example 1 using intermediate 7. LCMS purity 94%, m/z 550 [M+H]⁺, ¹H NMR (300 MHz, CDCl₃), δ: 7.39-7.29 (2H, m), 6.96-6.92 (2H, m), 6.63 (2H, d, J=9.3 Hz), 5.82 (1H, d, J=9.9 Hz), 3.97 (2H, t, J=5.9 Hz), 3.33-3.29 (1H, m), 1.90-1.79 (3H, m), 1.66-1.60 (1H, m), 1.42 (9H, s)

### Example 7 Cyclopentyl (S)-2-Amino-5-{4-[6-amino-5-(4-difluorobenzoyl)-2-oxo-2H-pyridin-1-yl]-3,5-difluorophenoxy}pentanoate

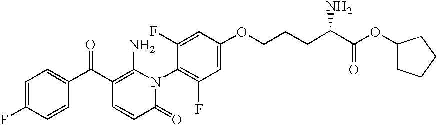

Example 7 was synthesised using similar methodology to Example 2 (and Intermediate 1) using 6-amino-5-(4-difluorobenzoyl)-1-(2,6-difluoro-4-hydroxy-phenyl)-1H-pyridin-2-one [prepared by methods described in WO03/076405].

LCMS purity 97%, m/z 544 [M+H]⁺, ¹H NMR (300 MHz, CDCl₃), δ: 7.52-7.61 (3H, m), 7.17 (2H, t, J=8.7 Hz), 6.71 (2H, d, J=9.4 Hz), 5.89 (1H, d, J=9.8 Hz), 5.19-5.28 (1H, m), 4.00-4.09 (2H, m), 3.43-3.65 (1H, m), 1.53-2.01 (12H, m).

### Example 8 Cyclopentyl 4-(2-{4-[6-amino-5-(2,4-difluorobenzoyl)-2-oxopyridin-1(2H)-yl]-3,5-difluorophenoxy}ethyl)piperazine-2-carboxylate

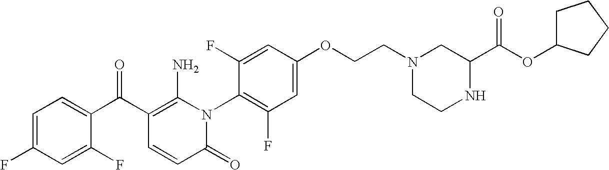

Example 8 was synthesised using the same methodology as Example 1 using intermediate 9. LCMS purity 90%, m/z 603 [M+H]⁺, ¹H NMR (300 MHz, CDCl₃), δ: 7.34-7.48 (2H, m), 6.88-7.06 (2H, m), 6.68-6.76 (2H, m), 5.90 (1H, d, J=9.8 Hz), 5.19-5.28 (1H, m), 3.59 (1H, dd, J=7.9, 3.2 Hz), 2.39-3.17 (9H, m), 1.54-1.97 (9H, m).

### Example 9 Cyclopentyl 4-[3-(4-{6-amino-5-[(2,4-difluorophenyl)carbonyl]-2-oxopyridin-1(2H)-yl}-3,5-difluorophenoxy)propyl]piperazine-2-carboxylate

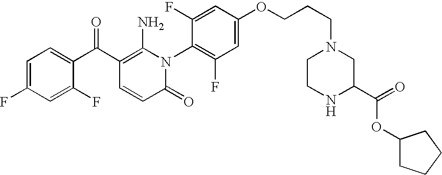

Example 9 was synthesised using the same methodology as Example 8 and using 3-bromopropanol in the synthesis of intermediate 9a. LCMS purity 98%, m/z 617 [M+H]⁺, ¹H NMR (300 MHz, CDCl₃), δ: 7.34-7.48 (2H, m), 6.88-7.05 (2H, m), 6.67-6.76 (2H, m), 5.90 (1H, d, J=9.8 Hz), 5.23 (1H, t, J=5.9 Hz), 4.13-4.17 (1H, m), 3.58 (1H, dd, J=7.7, 3.0 Hz), 3.05-3.16 (1H, m), 2.85-2.96 (2H, m), 2.42-2.70 (4H, m), 2.28-2.37 (1H, m), 1.96-2.07 (4H, m), 1.82-1.95 (1H, m), 1.54-1.79 (7H, m).

### Example 10 Cyclopentyl 4-[2-(4-{6-amino-5-[(4-fluorophenyl)carbonyl]-2-oxopyridin-1(2H)-yl}phenyl)ethyl]piperazine-2-carboxylate

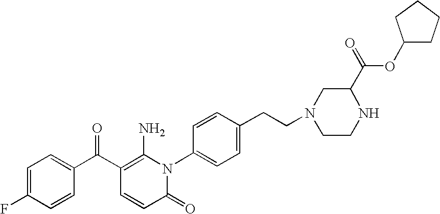

Example 10 was synthesised using the same methodology as Example 1 using intermediate 11. LCMS purity 90%, m/z 533 [M+H]⁺, ¹H NMR (300 MHz, MeOD), δ: 7.75-7.54 (5H, m), 7.29-7.23 (4H, m), 5.83 (1H, d, J=9.6 Hz), 5.25 (1H, m), 3.57 (1H, m), 3.12-2.71 (6H, m), 2.47 (1H, m), 2.36 (1H, m), 1.96-1.65 (8H, m), 1.43 (2H, m).

### Example 11 (S)-4-{4-[6-Amino-5-(2,4-difluorobenzoyl)-2-oxo-2H-pyridin-1-yl]-3,5-difluorophenoxy}-2-N-cyclohexylaminobutanoic Acid Hydrochloride

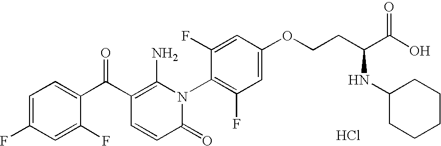

To a solution of the compound of Example 5 (20 mg, 0.0317 mmol) in a mixture of MeOH (0.3 ml) and THF (0.3 ml) was added 2M aq NaOH (0.3 ml). The reaction mixture was allowed to stand at RT for 3 h. Upon completion the reaction mixture was evaporated to dryness by blowing under a flow of N₂, acidified to pH 1-2 by dropwise addition of 2M aq HCl. The resulting white solid formed was collected by filtration. Yield=9 mg, 48%.: LCMS purity 98%, m/z 562 [M+H]⁺, ¹H NMR (400 MHz, MeOD), δ: 1.00-1.45 (5H, m), 1.55-1.60 (1H, m), 1.75-1.85 (2H, m), 2.00-2.15 (2H, m), 2.20-2.35 (2H, m), 2.95-3.10 (1H, m), 3.75-3.85 (1H, m), 4.10-4.30 (2H, m), 5.70 (1H, d), 6.75-6.85 (2H, m), 7.00-7.10 (2H, m), 7.35-7.45 (2H, m).

The following examples were prepared in a similar manner to Example 11

### Example 12 (S)-2-Amino-5-{4-[6-amino-5-(2,4-difluorobenzoyl)-2-oxo-2H-pyridin-1-yl]-3,5-difluorophenoxy}pentanoic Acid Trifluoroacetate

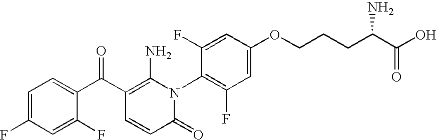

From the compound of Example 2. LCMS purity 97%, m/z 494 [M+H]⁺, ¹H NMR (400 MHz, MeOD), δ: 1.80-2.10 (4H, m), 3.90-4.00 (1H, m), 4.00-4.10 (2H, m), 5.65 (1H, d), 6.75-6.80 (2H, m), 6.95-7.05 (2H, m), 7.30-7.45 (2H, m).

### Example 13 (R)-2-Amino-4-{4-[6-amino-5-(2,4-difluorobenzoyl)-2-oxo-2H-pyridin-1-yl]-3,5-difluorophenoxy}butyric Acid

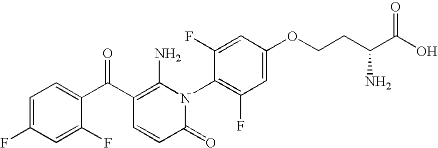

From the compound of Example 4. LCMS purity 97%, m/z 480 [M+H]⁺, ¹H NMR (400 MHz, MeOD), δ: 2.35-2.55 (2H, m), 4.15-4.20 (1H, m), 4.25-4.35 (2H, m), 5.75 (1H, d), 6.85-7.00 (2H, m), 7.05-7.20 (2H, m), 7.40-7.55 (2H, m).

### Example 14 (S)-2-Amino-4-{4-[6-amino-5-(2,4-difluorobenzoyl)-2-oxo-2H-pyridin-1-yl]-3,5-difluorophenoxy}butyric Acid

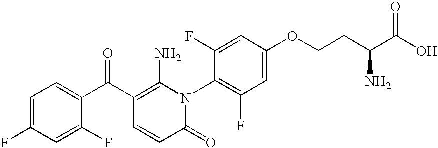

From the compound of Example 3, LCMS purity 97%, m/z 480 [M+H]⁺, ¹H NMR (400 MHz, MeOD), δ: 2.35-2.55 (2H, m), 4.15-4.20 (1H, m), 4.25-4.35 (2H, m), 5.75 (1H, d), 6.85-7.00 (2H, m), 7.05-7.20 (2H, m), 7.40-7.55 (2H, m).

### Example 15 (S)-4-{4-[6-Amino-5-(2,4-difluorobenzoyl)-2-oxo-2H-pyridin-1-yl]-3,5-difluorophenoxy}-2-tert-butoxycarbonylaminobutyric Acid

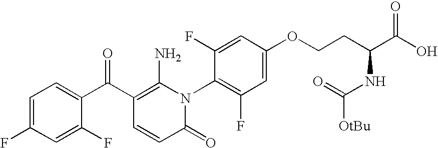

From intermediate 3, LCMS purity 98%, m/z 580 [M+H]⁺, ¹H NMR (400 MHz, MeOD), δ: 1.35 (9H, s), 2.00-2.10 (1H, m), 2.20-2.35 (1H, m), 4.05-4.15 (2H, m), 4.20-4.30 (1H, m), 5.70 (1H, d), 6.75-6.85 (2H, m), 7.00-7.10 (2H, m), 7.35-7.50 (2H, m).

### Example 16 (S)-2-Amino-5-{4-[6-amino-5-(4-difluorobenzoyl)-2-oxo-2H-pyridin-1-yl]-3,5-difluorophenoxy}pentanoic Acid

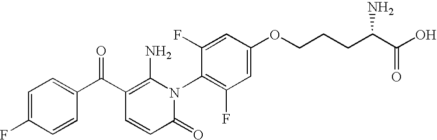

From the compound of Example 7. LCMS purity 98%, m/z 476 [M+H]⁺, ¹H NMR (300 MHz, DMSO), δ: 7.49-7.63 (3H, m), 7.33 (2H, t, J=8.5 Hz), 7.05 (2H, d, J=10.0 Hz), 5.72 (1H, d, J=9.8 Hz), 4.06-4.15 (2H, m), 1.76-1.97 (4H, m).

### Example 17 4-(2-{4-[6-amino-5-(2,4-difluorobenzoyl)-2-oxopyridin-1(2H-yl]-3,5-difluorophenoxy}ethyl)piperazine-2-carboxylate

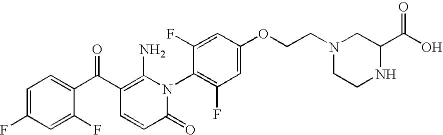

From the compound of Example 8. LCMS purity 90%, m/z 535 [M+H]⁺, ¹H NMR (300 MHz, MeOD), δ: 7.56-7.45 (2H, m), 7.12 (2H, t, J=8.6 Hz), 6.98-6.88 (2H, m), 5.80 (1H, d, J=9.6 Hz), 4.26 (2H, t, J=5.2 Hz), 3.71-3.49 (2H, m), 3.47-3.34 (1H, m), 3.26-3.01 (2H, m), 2.94 (2H, t, J=5.3 Hz), 2.60-2.48 (2H, m)

### Measurement of Biological Activity

p38 MAP Kinase activity

The ability of compounds to inhibit p38 MAPα Kinase activity was measured in an assay performed by Upstate (Dundee UK). In a final reaction volume of 25 μL, p38 MAP Kinase α (5-10 mU) is incubated with 25 mM Tris pH 7.5, 0.02 mM EGTA, 0.33 mg/ml myelin basic protein, 10 mM MgAcetate and [γ-³³P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

Duplicate data points are generated from a ⅓ log dilution series of a stock solution in DMSO. Nine dilutions steps are made from a top concentration of 10 μM, and a ‘no compound’ blank is included. The standard radiometric filter-binding assay is performed at an ATP concentration at, or close to, the Km. Data from scintillation counts are collected and subjected to free-fit analysis by Prism software. From the curve generated, the concentration giving 50% inhibition is determined and reported.

### LPS-Stimulation of THP-1 Cells

THP-1 cells were plated in 100 μl at a density of 4×10⁴ cells/well in V-bottomed 96 well tissue culture treated plates and incubated at 37° C. in 5% CO₂ for 16 hrs. 2 hrs after the addition of the inhibitor in 100 μl of tissue culture media, the cells were stimulated with LPS (E coli strain 005:B5, Sigma) at a final concentration of 1 μg/ml and incubated at 37° C. in 5% CO₂ for 6 hrs. TNF-α levels were measured from cell-free supernatants by sandwich ELISA (R&D Systems #QTA00B)

### LPS-Stimulation of Human Whole Blood

Whole blood was taken by venous puncture using heparinised vacutainers (Becton Dickinson) and diluted in an equal volume of RPMI 1640 tissue culture media (Sigma). 100 μl was plated in V-bottomed 96 well tissue culture treated plates. 2 hrs after the addition of the inhibitor in 100 μl of RPMI 1640 media, the blood was stimulated with LPS (E. coli strain 005:B5, Sigma) at a final concentration of 100 ng/ml and incubated at 37° C. in 5% CO₂ for 6 hrs. TNF-α levels were measured from cell-free supernatants by sandwich ELISA (R&D Systems #QTA00B)

IC50 values were allocated to one of three ranges as follows:

### Range A: IC50<100 nM

Range B: 100 nM<IC50<1000 nM

### Range C: IC50>1000 nM

NT=not tested

| | | | Inhibitor activity |
|---|---|---|---|
| | Inhibitor activity | Inhibitor activity | versus human |
| | versus p38 | versus THP-1 | whole blood |
| Example | MAPKa | TNFα release | TNFα release |
| Intermediate 1 | A | B | NT |
| Intermediate 2 | A | B | NT |
| 1 | A | A | A |
| 2 | A | A | A |
| 3 | A | A | A |
| 4 | A | A | B |
| 5 | A | A | NT |
| 6 | A | A | B |
| 7 | A | A | B |
| 8 | A | A | NT |
| 9 | NT | NT | NT |
| 10 | A | A | NT |
| 11 | A | NT | NT |
| 12 | A | NT | NT |
| 13 | A | NT | NT |
| 14 | A | NT | NT |
| 15 | A | NT | NT |
| 16 | A | NT | NT |
| 17 | A | NT | NT |

### Broken Cell Carboxylesterase Assay

Any given compound of the present invention wherein R₁ is an ester group may be tested to determine whether it meets the requirement that it be hydrolysed by intracellular esterases, by testing in the following assay.

### Preparation of Cell Extract

U937 or Hut78 tumour cells (˜10⁹) were washed in 4 volumes of Dulbeccos PBS (˜1 litre) and pelleted at 525 g for 10 min at 4° C. This was repeated twice and the final cell pellet was resuspended in 35 ml of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at room temperature by LCMS (Sciex API 3000, HP1100 binary pump, CTC PAL). Chromatography was based on an AceCN (75×2.1 mm) column and a mobile phase of 5-95% acetonitrile in water/0.1% formic acid.

Rates of hydrolysis are expressed in pg/mL/min.

Table 1 presents data showing that several amino acid ester motifs, conjugated to various intracellular enzyme inhibitors by several different linker chemistries are all hydrolysed by intracellular carboxyesterases to the corresponding acid.

TABLE 1

| | | | | Preparation of |
|---|---|---|---|---|
| | | | Hydrolysis Rate Range | amino ester |
| Structure of amino acid ester conjugate | R | Linker | U937Cells (pg/mL/min) | conjugate |
| 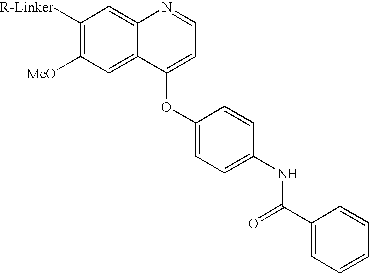 | 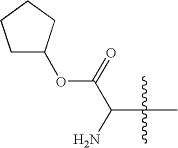 | 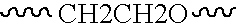 | 100-1000 | WO2006117552 |
| 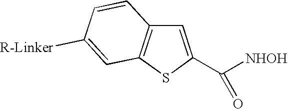 | 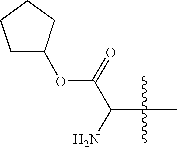 | 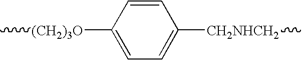 | 1000-50000 | WO2006117548 |
| 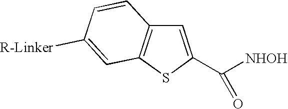 | 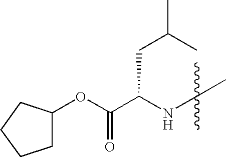 | 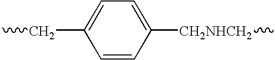 | >50000 | WO2006117549 |
| 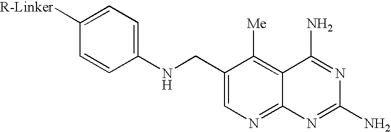 | 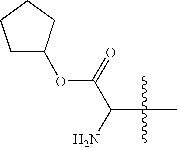 | 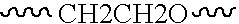 | >50000 | WO2006117567 |
| 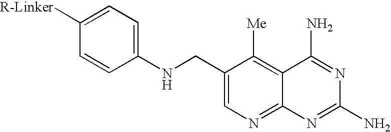 | 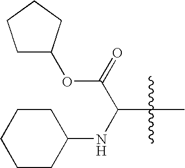 | 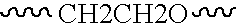 | 1000-50000 | WO2006117567 |
| 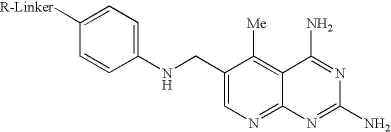 | 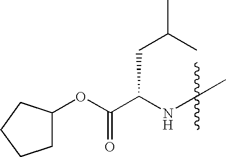 | 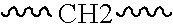 | 1000-50000 | WO2006117567 |
| 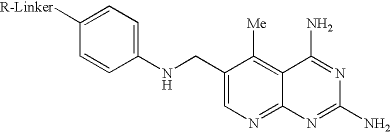 | 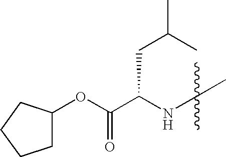 | 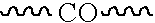 | >50000 | WO2006117567 |
| 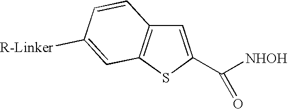 | 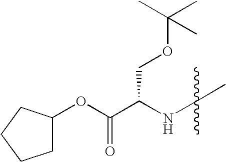 | 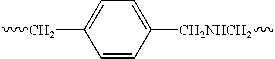 | >50000 | WO2006117549 |
| 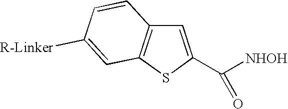 | 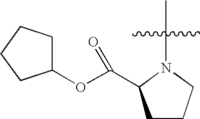 | 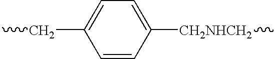 | >50000 | WO2006117549 |

1. A compound of formula (I): 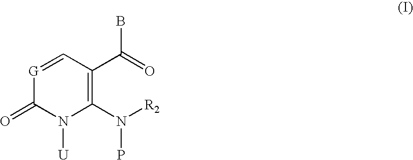
wherein:
G is —N═ or —CH═
B is an optionally substituted divalent mono- or bicyclic aryl or heteroaryl radical having 5-13 ring members;
R₂ is hydrogen or optionally substituted C₁-C₃ alkyl;
P represents hydrogen and U represents a radical of formula (IA); or U represents hydrogen and P represents a radical of formula (IA);
-A-(CH₂)_{z}-L¹-Y¹—R (IA)
wherein
A represents an optionally substituted divalent mono- or bicyclic carbocyclic or heterocyclic radical having 5-13 ring members;
z is 0 or 1;
Y¹ is a bond, —(C═O)—, —S(O₂)—, (C═O)NR₃—, —NR₃(C═O)—, —S(O₂)NR₃—, —NR₃S(O₂)—, or —NR₃(C═O)NR₅—, wherein R₃ and R₅ are independently hydrogen or optionally substituted (C₁-C₆)alkyl,
L¹ is a divalent radical of formula -(Alk₁)ₘ(Q)ₙ(Alk²)ₚ— wherein
m, n and p are independently 0 or 1,
Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
R is a radical of formula (X) or (Y) 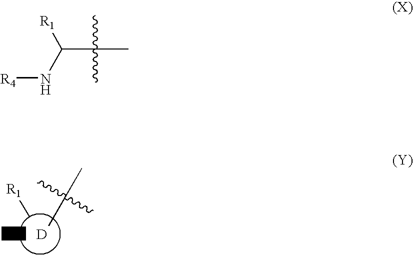
wherein
R₁ is a carboxylic acid group (—COOH), or an ester group which is hydrolysable by one or more intracellular carboxylesterase enzymes to a carboxylic acid group;
R₄ is hydrogen; or optionally substituted C₁-C₆ alkyl, C₃-C₇ cycloalkyl, aryl or heteroaryl or —(C═O)R₃, —(C═O)OR₃, or —(C═O)NR₃ wherein R₃ is hydrogen or optionally substituted (C₁-C₆)alkyl; and
D is a monocyclic heterocyclic ring of 5 or 6 ring atoms wherein R₁ is linked to a ring carbon adjacent the ring nitrogen shown, and ring D is optionally fused to a second carbocyclic or heterocyclic ring of 5 or 6 ring atoms in which case the bond shown intersected by a wavy line may be from a ring atom in said second ring.

2. A compound as claimed in claim 1 wherein B is optionally substituted phenyl, or pyridinyl

3. A compound as claimed in claim 1 wherein R₂ is hydrogen or methyl.

4. A compound as claimed in claim 1 wherein P is hydrogen and U is a radical of formula (IA) as defined in claim 1.

5. A compound as claimed in wherein A is optionally substituted 1,4 phenylene or selected from those of formulae A-X, optionally substituted: 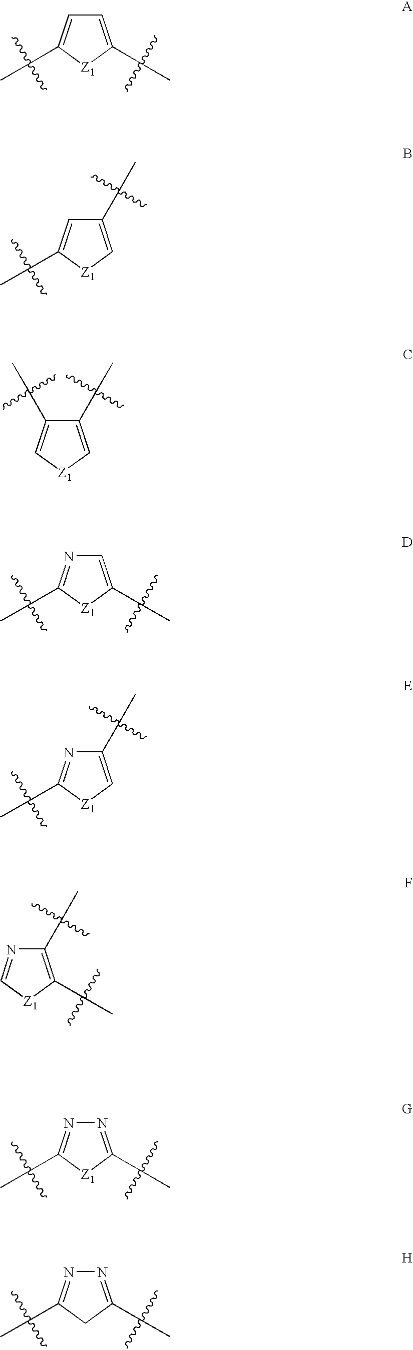 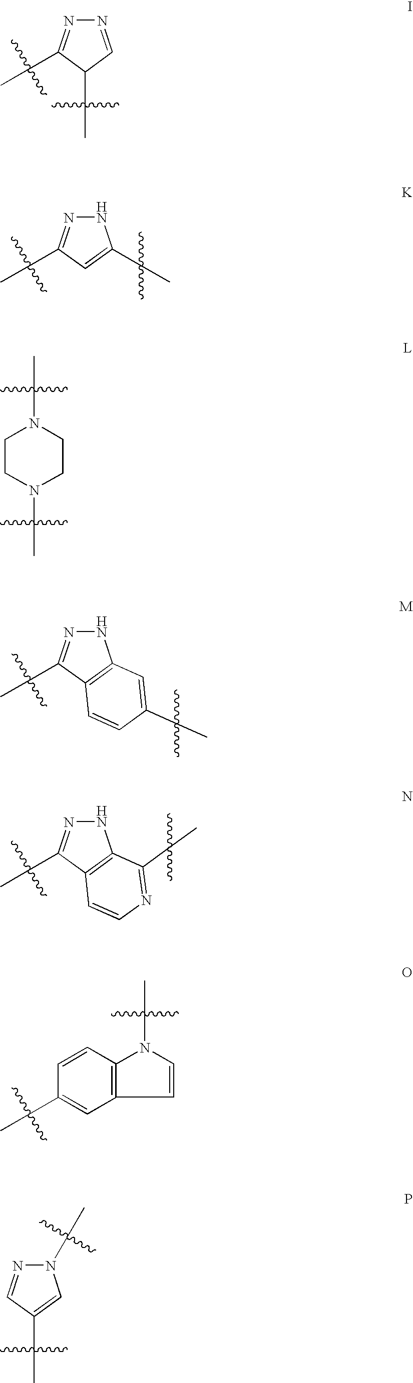 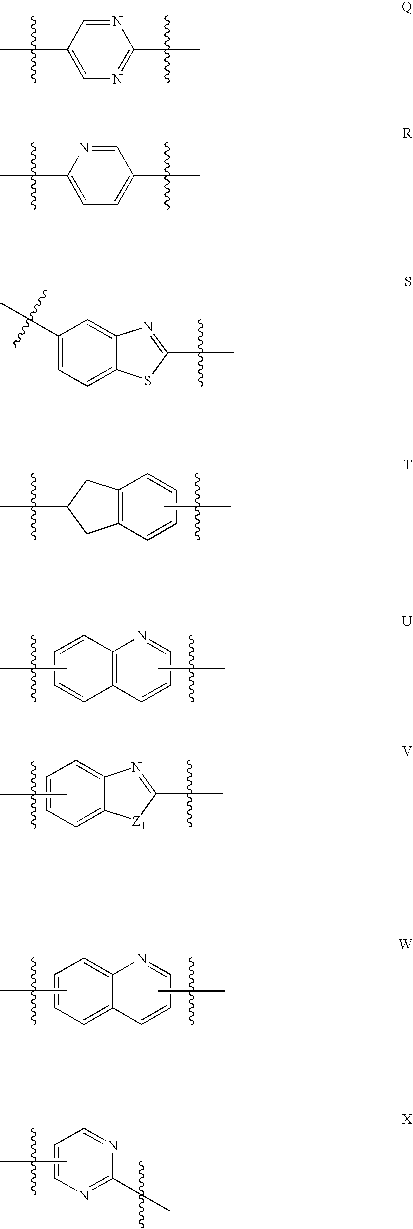
wherein Z₁ is NH, S or O.

6. A compound as claimed in claim 1 which has formula (IIA), (IIB) and (IIC): 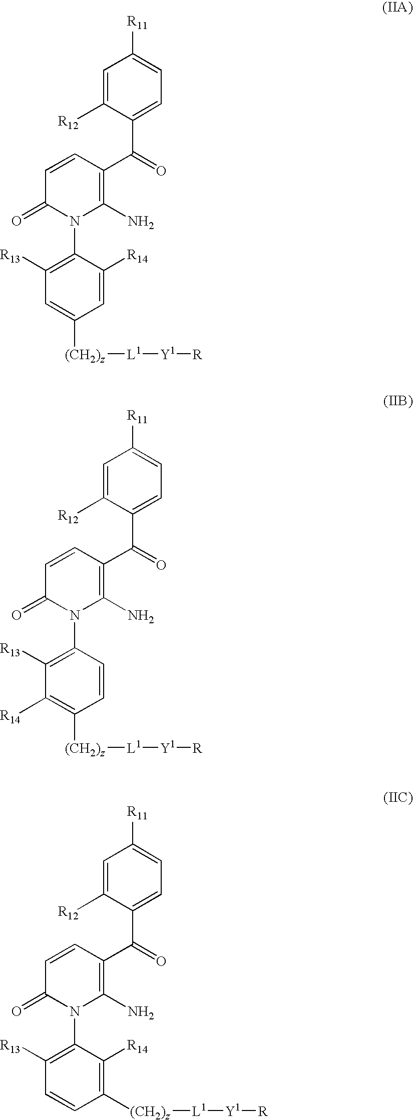
wherein
R₁₁═F, R₁₂═H, R₁₃═H and R₁₄═H; or
R₁₁═F, R₁₂═F, R₁₃═H and R₁₄═H; or
R₁₁═F, R₁₂═H, R₁₃═F and R₁₄═F; or
R₁₁═F, R₁₂═F, R₁₃═F and R₁₄═F; or
R₁₁═F, R₁₂═F, R₁₃═F and R₁₄═H.
and wherein z, L¹, Y¹, and R are as defined in claim 1.

7. A compound as claimed in claim 1 wherein z is 0.

8. A compound as claimed in claim 1 wherein Y¹ is —C(═O)NR₃— or —NR₃C(═O)— wherein R₃ is hydrogen or optionally substituted C₁-C₆ alkyl.

9. A compound as claimed in claim 1 wherein Y¹ is a bond.

10. A compound as claimed in claim 1 wherein, in the radical L¹, Alk¹ and Alk², when present, are selected from —CH₂—, CH₂CH₂—, —CH₂CH₂CH₂—, and divalent cyclopropyl, cyclopentyl and cyclohexyl radicals.

11. A compound as claimed in claim 1 wherein, in the radical L¹, m and p are 0.

12. A compound as claimed in claim 1 wherein, in the radical L¹, n and p are 0 and m is 1.

13. A compound as claimed in claim 1 wherein, in the radical L¹, m, n and p are all 0.

14. A compound as claimed in claim 1 wherein the radical
—Y¹-L¹-[CH₂]_{z}— is selected from —(CH₂)₃NH—, —CH₂C(═O)NH—, —CH₂CH₂C(═O)NH—, —CH₂C(O)O—, —CH₂S—, —CH₂CH₂C(O)O—, —(CH₂)₄NH—, —CH₂CH₂S—, —CH₂O, —CH₂CH₂O—, —CH₂CH₂CH₂O—

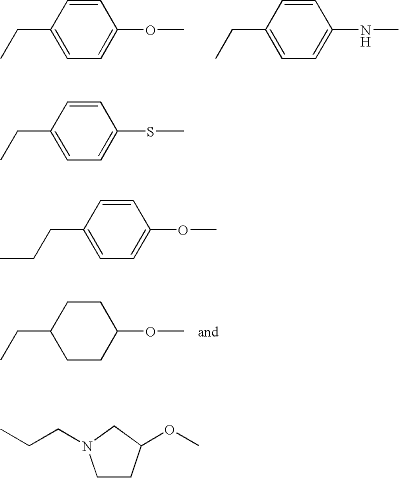

15. A compound as claimed in claim 1 wherein the radical —Y¹-L¹-[CH₂]_{z}— is —CH₂—.

16. A compound as claimed in claim 1 wherein the radical —Y¹-L¹-[CH₂]_{z}— is —CH₂CH₂O— or —CH₂CH₂CH₂O—.

17. A compound as claimed in claim 1 wherein in the group R, R₁ is an ester group of formula —(C═O)OR₇
wherein R₇ is R₈R₉R₁₀C— wherein
(i) R₈ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen.

18. A compound as claimed in claim 16 wherein R₇ is methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl or methoxyethyl.

19. A compound as claimed in claim 16 wherein R₇ is cyclopentyl.

20. A compound as claimed in claim 1 wherein R is a group of formula (X) as defined in claim 1.

21. A compound as claimed in claim 20 wherein R₄ is optionally substituted C₁-C₆ alkyl, C₃-C₇ cycloalkyl, phenyl, phenyl(C₁-C₆ alkyl)-, or —(C═O)R₃, wherein R₃ is optionally substituted C₁-C₆ alkyl, C₃-C₇ cycloalkyl, phenyl, phenyl(C₁-C₆ alkyl)-, or —(C═O)R₃, wherein R₃ is phenyl, phenyl(C₁-C₆ alkyl)-.

22. A compound as claimed in claim 20 wherein R₄ is methyl, ethyl, n- or isopropyl, n-, iso- or sec-butyl, cyclopropyl, cyclopentyl, cyclohexyl, phenyl, benzyl, acetyl, thienylcarbonyl, benzoyl, 4-methoxybenzoyl, pyridyl, pyridylmethyl, or pyridylcarbonyl.

23. A compound as claimed in claim 20 wherein R₄ is hydrogen, —(C═O)R₃, —(C═O)OR₃, or —(C═O)NHR₃ wherein R₃ is hydrogen or optionally substituted (C₁-C₆)alkyl.

24. A compound as claimed in claim 1 wherein R is a group of formula (Y).

25. A compound as claimed in claim 24 wherein ring or ring system D is selected from the following:

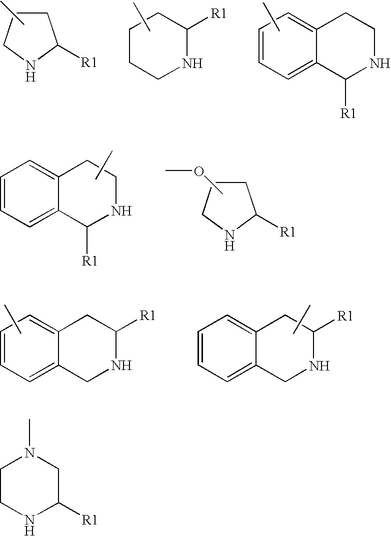

26. A compound as claimed in claim 1 having the structure of any of the compounds of the specific Examples herein.

27. A compound as claimed in claim 1 which is in the form of a pharmaceutically acceptable salt.

28. A pharmaceutical composition comprising a compound as claimed in claim 1, together with a pharmaceutically acceptable carrier.

29. (canceled)

30. The use of a compound as claimed in any of claims 1 to 27 in the preparation of a composition for the treatment of autoimmune or inflammatory disease.

31. A method of inhibiting the activity of a p38 MAP kinase enzyme comprising contacting the enzyme with an amount of a compound as claimed in claim 1 effective for such inhibition.

32. A method for the treatment of autoimmune or inflammatory disease which comprises administering to a subject suffering such disease an effective amount of a compound as claimed in claim 1.

33. The method as claimed in claim 32 wherein the disease is psoriasis, inflammatory bowel disease, Crohns disease, ulcerative colitis, chronic obstructive pulmonary disease, asthma, multiple sclerosis, diabetes, atopic dermatitis, graft versus host disease, or systemic lupus erythematosus.

34. The method as claimed in claim 32 wherein the disease is rheumatoid arthritis.
